(19) United States
(12) Patent Application Publication
(10) Pub. No.: US 2010/0287657 A1
(43) Pub. Date: Nov. 11, 2010

(54) Novel Nucleotide Sequences Encoding Nicotiana Beta-1,2-Xylosyltransferase

(75) Inventor: Koen Weterings, Zingem (BE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Bayer BioScience N.V., Gent (BE)

(21) Appl. No.: 12/293,761

(22) PCT Filed: Mar. 15, 2007
(86) PCT No.: PCT/EP2007/002322

§ 371 Date: Jul. 19, 2010

Related U.S. Application Data
(60) Provisional application No. 60/787,551, filed on Mar. 30, 2006.

(30) Foreign Application Priority Data
Mar. 23, 2006 (EP) 06075714.3

(51) Int. Cl. A01H 5/00; C12N 15/82; C12Q 1/68; C07H 21/04; C12N 5/10

(52) U.S. Cl. 800/278; 800/317.3; 435/468; 435/6; 536/23.6; 435/419

(57) ABSTRACT

Provided are novel β1,2-xylosyltransferase nucleotide sequences and uses thereof.

The following invention relates to novel nucleotide sequences from Nicotiana species and cultivars, particularly from Nicotiana benthamiana and Nicotiana tabacum cv. Petite Havana SR1, encoding β1,2-xylosyltransferase (XylT) and their use to produce modified Nicotiana plants, particularly Nicotiana benthamiana and Nicotiana tabacum cv. Petite Havana SR1 plants, which have a lower level or altered pattern of immunogenic protein-bound N-glycans, particularly a lower level of beta-1,2-xylose residues on the protein-bound N-glycans, than counterpart unmodified Nicotiana plants. Such Nicotiana plants may be obtained by lowering the expression of the endogenous Nicotiana XylT gene(s), e.g., by modifying the activity of endogenous Nicotiana XylT gene(s), by exchanging the endogenous Nicotiana XylT gene for another allele of the XylT gene which provides a lower level of beta-1,2-xylose residues on the protein-bound N-glycans, or by any combination thereof.

### DESCRIPTION OF RELATED ART

The use of transgenic plants for the production of value-added recombinant proteins, such as antibodies, vaccines, human blood products, hormones, growth regulators and the like, is described to offer many practical, economic and safety advantages compared with more conventional systems such as animal and insect cell cultures, yeast, filamentous fungi and bacteria (reviewed by Stoger et al., 2002; Twyman et al., 2003; Fischer et al., 2004).

Although the protein synthesis pathway is largely the same in plants and animals, there are some differences in posttranslational modifications, particularly with respect to glycan-chain structures. Thus, plant-derived recombinant human proteins tend to have the carbohydrate groups beta(1→2)-xylose and alpha(1→3)-fucose, which are absent in mammals, but lack the terminal galactose and sialic acid residues that are found on many native human glycoproteins (Twyman et al., 2003).

The enzyme that catalyses the transfer of xylose from UDP-xylose to the core beta-linked mannose of protein-bound N-glycans is beta-1,2-xylosyltransferase (XylT). XylT is an enzyme unique to plants and some non-vertebrate animal species, e.g. in Schistosoma species (Khoo et al., 1997) and snail (e.g. Mulder et al., 1995) and does not occur in human beings or in other vertebrates.

Tezuka et al. (1992) characterized a XylT of sycamore (Acer pseudoplatanus L.).

Zeng et al. (1997) described the purification of a XylT from soybean microsomes. Only a part of the soybean XylT cDNA was isolated (WO99/29835 A1).

Strasser et al. (2000) and WO01/64901 describe the isolation of an Arabidopsis XylT gene, the predicted amino acid sequence of the encoded XylT protein and its enzymatic activity in vitro and in vivo.

The following database entries identifying experimentally demonstrated and putative XylT cDNA and gene sequences, parts thereof or homologous sequences, could be identified: AJ627182, AJ627183 (Nicotiana tabacum cv. Xanthi), AM179855 (Solanum tuberosum), AM179856 (Vitis vinifera), AJ891042 (Populus alba×Populus tremula), AY302251 (Medicago sativa), AJ864704 (Saccharum officinarum), AM179857 (Zea mays), AM179853 (Hordeum vulgare), AM179854 (Sorghum bicolor), BD434535, AJ277603, AJ272121, AF272852, AX236965 (Arabidopsis thaliana), AJ621918 (Oryza saliva), AR359783, AR359782, AR123000, AR123001 (Soybean), AJ618933 (Physcomitrella patens).

Strasser et al. (2004a) report on two approaches for the modulation of the N-glycosylation pathway in plants: First posttranscriptional gene silencing was used to knock down the expression of beta-1,2-N-acetylglucosaminyltransferase I (GnTI), an enzyme involved in the processing of oligomannosidic residues to hybrid and complex N-glycans in higher eukaryotes, to assess the influence of GnTI expression on the formation of complex N-glycans in Nicotiana benthamiana. N-glycan profiling revealed no significant changes of the total N-glycan pattern, indicating that even a minor residual activity of GnTI allows the biosynthesis of complex N-glycans in Nicotiana benthamiana. They further report that a similar approach for the knock down of XylT resulted in a significant reduction of beta-1,2-xylosylated N-glycans. Second, in order to achieve a complete elimination of beta-1,2-xylose and alpha-1,3-fucose residues from N-glycans, triple knock out Arabidopsis plants were generated using insertion mutation lines. These plants exhibit complete deficiency of active beta-1,2-xylosyltransferase and core alpha-1,3-fucosyltransferase, lack immunogenic protein-bound N-glycans and synthesize predominantly humanized structures with terminal beta-N-acetylglucosamine residues (Strasser et al., 2004b).

Leafy crops, such as tobacco, are considered to be strong candidates for the commercial production of recombinant proteins (see e.g. Twyman et al., 2003).

The aim of the current invention is to provide alternative XylT cDNA and gene sequences from Nicotiana species and cultivars, particularly from Nicotiana benthamiana and Nicotiana tabacum cv. Petite Havana SR1, which are better suited to modify the expression of XylT in particular Nicotiana species or cultivars.

### SUMMARY OF THE INVENTION

In one aspect of the invention, a method is provided to produce a Nicotiana plant cell or plant having a low level of beta-1,2-xylose residues on protein-bound N-glycans comprising the steps of introducing a chimeric gene into plant cells of a Nicotiana species or cultivar to generate transgenic plant cells, the chimeric gene comprising operably linked a plant expressible promoter; a transcribable DNA region comprising a first sense DNA region comprising a nucleotide sequence of at least 19 out of 20 consecutive nucleotides selected from a nucleotide sequence encoding a Nicotiana XylT protein, or the complement thereof, the nucleotide sequence preferably obtainable from the Nicotiana species or cultivar, wherein the at least 19 out of 20 consecutive nucleotides encode at least one Nicotiana species- or cultivar-specific XylT amino acid, or selected from a nucleotide sequence of a Nicotiana XylT gene or a Nicotiana XylT cDNA, or the complement thereof, the nucleotide sequence preferably obtainable from the Nicotiana species or cultivar, wherein the at least 19 out of 20 consecutive nucleotides comprise at least one Nicotiana species-specific XylT nucleotide; a second antisense DNA region comprising a nucleotide sequence of at least 19 consecutive nucleotides which have at least 95% sequence identity to the complement of the first DNA region; wherein an RNA molecule transcribed from the transcribable DNA region is capable of forming a double stranded RNA region at least between an RNA region transcribed from the first sense DNA region and an RNA region transcribed from the second antisense DNA region; and a DNA region comprising a transcription termination and polyadenylation signal functional in plants; optionally, identifying a transgenic Nicotiana plant cell which has a lower level of beta-1,2-xylose residues on protein-bound N-glycans than an untransformed Nicotiana plant cell; optionally, regenerating the transgenic Nicotiana plant cells to obtain transgenic Nicotiana plants; and optionally, identifying a transgenic Nicotiana plant which has a lower level of beta-1,2-xylose residues on protein-bound N-glycans than an untransformed Nicotiana plant. The Nicotiana species- or cultivar-specific XylT amino acid or nucleotide may be a Nicotiana benthamiana-specific or Nicotiana tabacum cv. Petite Havana SR1-specific XylT amino acid or nucleotide and the Nicotiana species or cultivar may preferably be Nicotiana benthamiana or Nicotiana tabacum cv. Petite Havana SR1, respectively. The nucleotide sequence encoding a Nicotiana XylT protein may comprise a nucleotide sequence encoding the amino acid sequence of SEQ ID No.: 12 or SEQ ID No.:14 or the amino acid sequence of SEQ ID No.: 4, SEQ ID No.:6, SEQ ID No.: 8 or SEQ ID No.:10, and the nucleotide sequence of the Nicotiana XylT gene may comprise the nucleotide sequence of SEQ ID No.: 11, SEQ ID No.:13, or SEQ ID No. 21, or the nucleotide sequence of SEQ ID No.: 3, SEQ ID No.: 5, SEQ ID No.: 8, SEQ ID No.:10, or SEQ ID No.: 17.

It is another object of the invention to provide a method to produce a Nicotiana plant cell or plant having a low level of beta-1,2-xylose residues on protein-bound N-glycans comprising the steps of providing one or more double stranded RNA molecules to plant cells or plants of a Nicotiana species or cultivar, wherein the double stranded RNA molecules comprise two RNA strands, one RNA strand consisting essentially of an RNA nucleotide sequence of 19 out of 20 to 21 consecutive nucleotides selected from a nucleotide sequence encoding a Nicotiana XylT protein, or the complement thereof, the nucleotide sequence preferably obtainable from the Nicotiana species or cultivar, wherein the 19 out of 20 to 21 consecutive nucleotides encode at least one Nicotiana species- or cultivar-specific XylT amino acid, or selected from the nucleotide sequence of a Nicotiana XylT gene or a Nicotiana XylT cDNA, or the complement thereof, the nucleotide sequence preferably obtainable from the Nicotiana species or cultivar, wherein the 19 out of 20 to 21 consecutive nucleotides comprise at least one Nicotiana species- or cultivar-specific XylT nucleotide; and identifying a Nicotiana plant cell or plant comprising the double stranded RNA molecule or molecules which has a lower level of beta-1,2-xylose residues on protein-bound N-glycans than a same Nicotiana plant cell or plant which does not comprise the double stranded RNA molecule or molecules. The double stranded RNA may be provided to the plant cells or plants by integrating a chimeric gene into the genome of plant cells of the Nicotiana species or cultivar to generate transgenic plant cells and, optionally, regenerating the plant cells to obtain transgenic plants, the chimeric gene comprising a DNA region comprising at least 19 out of 20 consecutive nucleotides selected from a nucleotide sequence encoding a Nicotiana XylT protein, or the complement thereof, the nucleotide sequence preferably obtainable from the Nicotiana species or cultivar, wherein the 19 out of 20 consecutive nucleotides encode at least one Nicotiana species- or cultivar-specific XylT amino acid, or selected from the nucleotide sequence of a Nicotiana XylT gene or a Nicotiana XylT cDNA, or the complement thereof, the nucleotide sequence preferably obtainable from the Nicotiana species or cultivar, wherein the 19 out of 20 consecutive nucleotides comprise at least one Nicotiana species-specific XylT nucleotide, in antisense and/or sense orientation; operably linked to a plant expressible promoter and a DNA region comprising a transcription termination and polyadenylation signal functional in plants. The Nicotiana species- or cultivar-specific XylT amino acid or nucleotide may be a Nicotiana benthamiana-specific or Nicotiana tabacum cv. Petite Havana SR1-specific XylT amino acid or nucleotide and the Nicotiana species or cultivar may preferably be Nicotiana benthamiana or Nicotiana tabacum cv. Petite Havana SR1, respectively. The nucleotide sequence encoding a Nicotiana XylT protein may comprise a nucleotide sequence encoding the amino acid sequence of SEQ ID No.: 12 or SEQ ID No.:14 or the amino acid sequence of SEQ ID No.: 4, SEQ ID No.:6, SEQ ID No.: 8 or SEQ ID No.:10, and the nucleotide sequence of the Nicotiana XylT gene may comprise the nucleotide sequence of SEQ ID No. 11, SEQ ID No.:13, or SEQ ID No. 21, or the nucleotide sequence of SEQ ID No.: 3, SEQ ID No.: 5, SEQ ID No.: 8, SEQ ID No.:10, or SEQ ID No.: 17.

It is yet another object of the invention to provide a method to identify a Nicotiana XylT DNA fragment, comprising the steps of providing genomic DNA or cDNA obtainable from a Nicotiana species or cultivar; selecting a means from the following group: a DNA fragment comprising a nucleotide sequence encoding the amino acid sequence of SEQ ID No.: 4, SEQ ID No.:6, SEQ ID No.: 8, SEQ ID No.:10, SEQ ID No.: 12, or SEQ ID No.:14, for use as a probe; a DNA fragment comprising the nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21, for use as a probe; a DNA fragment or oligonucleotide comprising a nucleotide sequence consisting of between 20 to 1513 consecutive nucleotides selected from a nucleotide sequence encoding the amino acid sequence of SEQ ID No.: 4, or SEQ ID No.:6, for use as a probe; a DNA fragment or oligonucleotide comprising a nucleotide sequence consisting of between 20 to 3574 consecutive nucleotides selected from a nucleotide sequence encoding the amino acid sequence of SEQ ID No.: 8, SEQ ID No.:10, SEQ ID No.: 12, or SEQ ID No.:14 for use as a probe; a DNA fragment or oligonucleotide comprising a nucleotide sequence consisting of between 20 to 3574 consecutive nucleotides selected from a nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21 for use as a probe; an oligonucleotide sequence having a nucleotide sequence comprising between 20 to 200 consecutive nucleotides selected from a nucleotide sequence encoding the amino acid sequence of SEQ ID No.: 4, or SEQ ID No.:6, for use as a primer in a PCR reaction; an oligonucleotide sequence having a nucleotide sequence comprising between 20 to 200 consecutive nucleotides selected from a nucleotide sequence encoding the amino acid sequence of SEQ ID No.: 8, SEQ ID No.:10, SEQ ID No.: 12, or SEQ ID No.:14, for use as a primer in a PCR reaction; an oligonucleotide sequence having a nucleotide sequence comprising between 20 to 200 consecutive nucleotides selected from the nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID NO.: 21, for use as a primer in a PCR reaction; or an oligonucleotide having the nucleotide sequence of any one of SEQ ID No.: 1, SEQ ID No.: 2, SEQ ID No.: 15 or SEQ ID No.: 16, SEQ ID No.:19 or SEQ ID No. 20 for use as a primer in a PCR reaction; and utilizing that means to identify a XylT DNA fragment from the Nicotiana species or cultivar by performing a PCR using the genomic DNA or the cDNA and the primers, or by performing hybridization using the genomic DNA or the cDNA and the probes. The identified fragment may subsequently be isolated and used to obtain a Nicotiana plant cell or plant having a low level of beta-1,2-xylose residues on protein-bound N-glycans.

The invention also provides a method to identify a Nicotiana XylT allele correlated with a low level of beta-1,2-xylose residues on protein-bound N-glycans comprising the steps of providing a population, optionally a mutagenized population, of different plant lines of a Nicotiana species or cultivar; identifying in each plant line of the population a Nicotiana XylT allele according to the method described above; analyzing the level of beta-1,2-xylose residues on protein-bound N-glycans of each plant line of the population and identifying those plant lines having a lower level of beta-1,2-xylose residues on protein-bound N-glycans than other plant lines; and correlating the low level of beta-1,2-xylose residues on protein-bound N-glycans in a plant line to the presence of a specific Nicotiana XylT allele. The Nicotiana XylT allele may be introduced into a Nicotiana plant cell or plant of choice to obtain a Nicotiana plant cell or plant with a low level of beta-1,2-xylose residues on protein-bound N-glycans.

It is yet another object of the invention to provide: an isolated DNA fragment encoding a protein comprising the amino acid sequence of SEQ ID No.: 12, or SEQ ID No.:14, or any part thereof encoding at least one Nicotiana benthamiana-specific XylT amino acid; an isolated DNA fragment comprising the nucleotide sequence of SEQ ID No.: 11, SEQ ID No.: 13, or SEQ ID No.: 21, or any part thereof comprising at least one Nicotiana benthamiana-specific XylT nucleotide; an isolated DNA fragment encoding a protein comprising the amino acid sequence of SEQ ID No.: 4 or SEQ ID No.:6, SEQ ID No.: 8, SEQ ID No.:10, or any part thereof encoding at least one Nicotiana tabacum cv. Petite Havana SR1-specific XylT amino acid; an isolated DNA fragment comprising the nucleotide sequence of SEQ ID No.: 3 or SEQ ID No.:5, SEQ ID No.: 7, SEQ ID No.:9, or SEQ ID No.: 17, or any part thereof comprising at least one Nicotiana tabacum cv. Petite Havana SR1-speck XylT nucleotide.

The invention further provides a chimeric gene comprising the following operably linked DNA fragments: a plant expressible promoter; a transcribable DNA region comprising a first DNA region comprising at least 19 out of 20 consecutive nucleotides selected from a nucleotide sequence encoding a Nicotiana XylT protein, or the complement thereof, wherein the 19 out of 20 consecutive nucleotides encode at least one Nicotiana species- or cultivar-specific XylT amino acid, or selected from the nucleotide sequence of a Nicotiana XylT gene or a Nicotiana XylT cDNA, or the complement thereof, wherein the 19 out of 20 consecutive nucleotides comprise at least one Nicotiana species-specific XylT nucleotide, in antisense orientation; a second DNA region comprising at least 19 out of 20 consecutive nucleotides selected from a nucleotide sequence encoding a Nicotiana XylT protein, or the complement thereof, wherein the 19 out of 20 consecutive nucleotides encode at least one Nicotiana species- or cultivar-specific XylT amino acid, or selected from the nucleotide sequence of a Nicotiana XylT gene or a Nicotiana XylT cDNA, or the complement thereof, wherein the 19 out of 20 consecutive nucleotides comprise at least one Nicotiana species-specific XylT nucleotide, in sense orientation, whereby an RNA molecule produced by transcription of the transcribable DNA region is capable of forming a double stranded RNA region by base-pairing at least between an RNA region corresponding to the first DNA region and an RNA region corresponding to the second DNA region; and a DNA region comprising a transcription termination and polyadenylation signal functional in plants. The chimeric gene may also comprise a plant expressible promoter; a DNA region comprising at least 19 out of 20 consecutive nucleotides selected from a nucleotide sequence encoding a Nicotiana XylT protein, or the complement thereof, wherein the 19 out of 20 consecutive nucleotides encode at least one Nicotiana species- or cultivar-specific XylT amino acid, or selected from the nucleotide sequence of a Nicotiana XylT gene or a Nicotiana XylT cDNA, or the complement thereof, wherein the 19 out of 20 consecutive nucleotides comprise at least one Nicotiana species-specific XylT nucleotide, in sense or antisense orientation; and a DNA region comprising a transcription termination and polyadenylation signal functional in plants.

Nicotiana plant cells comprising such chimeric genes and Nicotiana plants consisting essentially of such Nicotiana plant cells, as well as seed thereof are also provided by the invention.

The invention also relates to the use of a nucleotide sequence encoding a protein comprising the amino acid sequence of SEQ ID No.: 4, SEQ ID No.:6, SEQ ID No.; 8, SEQ ID No.: 10, SEQ ID No.: 12, or SEQ ID No.:14, or any part thereof comprising at least 19 out of 20 consecutive nucleotides encoding at least one Nicotiana species- or cultivar-specific XylT amino acid, to decrease the level of beta-1,2-xylose residues on protein-bound N-glycans in a Nicotiana plant, or the use of a nucleotide sequence comprising the nucleotide sequence of SEQ ID No.: 3, SEQ ID No.:5, SEQ ID No.: 7, SEQ ID No.:9, SEQ ID No.: II, SEQ ID No.: 13, SEQ ID No.: 17 or SEQ ID No.: 21, or any part thereof comprising at least 19 out of 20 consecutive nucleotides comprising at least one Nicotiana species- or cultivar-specific XylT nucleotide, to decrease the level of beta-1,2-xylose residues on protein-bound N-glycans in a Nicotiana plant, to identify a XylT gene or XylT cDNA in a Nicotiana species or cultivar, to identify an allele of a XylT gene correlated with a low level of beta-1,2-xylose residues on protein-bound N-glycans in a Nicotiana species or cultivar, or to introduce an allele of a XylT gene correlated with a low level of beta-1,2-xylose residues on protein-bound N-glycans in a Nicotiana species or cultivar.

With the foregoing and other objects, advantages and features of the invention that will become hereinafter apparent, the nature of the invention may be more clearly understood by reference to the following detailed description of different embodiments of the invention, the appended claims and the figures.

### BRIEF DESCRIPTION OF THE FIGURES

FIG. 1 is a global DNA alignment (based on the standard linear scoring matrix with following parameters: mismatch penalty=2, open gap penalty=4 and extend gap penalty=1) between a cDNA sequence of Nicotiana tabacum cv. Xanthi encoding a putative XylT protein (accession number AJ627182; SEQ ID NO:23) and two different XylT cDNA sequences isolated from Nicotiana tabacum cv. Petite Havana SR1 (SEQ ID No. 3 and 5). Dots represent nucleotides in the Nicotiana tabacum cv. Petite Havana SR1 cDNA sequences that are identical to the corresponding nucleotides in the Nicotiana tabacum cv. Xanthi cDNA sequence; dashes represent the absence of nucleotides in the Nicotiana tabacum cv. Petite Havana SR1 cDNA sequences corresponding to nucleotides in the Nicotiana tabacum cv. Xanthi cDNA sequence.

FIG. 2 is a global protein alignment (based on the blossum 62 scoring matrix) between the putative XylT protein encoded by the cDNA sequence from Nicotiana tabacum cv. Xanthi (accession number AJ627182; SEQ ID NO:24) and by the two different XylT cDNA sequences isolated from Nicotiana tabacum cv. Petite Havana SR1 (SEQ ID NO: 4 and 6). Dots represent amino acids in the Nicotiana tabacum cv. Petite Havana SR1 protein sequences that are identical to the corresponding amino acids in the Nicotiana tabacum cv. Xanthi protein sequence; dashes represent the absence of amino acids in the Nicotiana tabacum cv. Petite Havana SR1 protein sequences corresponding to amino acids in the Nicotiana tabacum cv. Xanthi protein sequence.

FIG. 3 is a global DNA alignment (based on the standard linear scoring matrix with following parameters: mismatch penalty=2, open gap penalty=4 and extend gap penalty=1) between the genomic DNA sequence from Nicotiana tabacum cv. Xanthi encoding a putative XylT protein (accession number AJ627183; SEQ ID NO:25) and two different XylT genomic DNA sequences isolated from Nicotiana tabacum cv. Petite Havana SR1 (SEQ ID NO: 7 and 9) and two different XylT genomic DNA sequences isolated from Nicotiana benthamiana (SEQ ID NO: 11 and 13). Dots represent nucleotides in the Nicotiana tabacum cv. Petite Havana SR1 genomic DNA sequences that are identical to the corresponding nucleotides in the Nicotiana tabacum cv. Xanthi genomic DNA sequence; dashes represent the absence of nucleotides in the Nicotiana tabacum cv. Petite Havana SR1 genomic DNA sequences corresponding to nucleotides in the Nicotiana tabacum cv. Xanthi genomic DNA sequence.

FIG. 4 is a global protein alignment (based on the blossum 62 scoring matrix) between the putative XylT protein encoded by the genomic DNA sequence from Nicotiana tabacum cv. Xanthi (accession number AJ627183; SEQ ID NO:26) and by the two different XylT genomic DNA sequences isolated from Nicotiana tabacum cv. Petite Havana SR1 (SEQ ID NO:8 and 10) and by the two different XylT genomic DNA sequences isolated from Nicotiana benthamiana (SEQ ID NO: 12 and 14). Dots represent amino acids in the Nicotiana tabacum cv. Petite Havana SR1 protein sequences that are identical to the corresponding amino acids in the Nicotiana tabacum cv. Xanthi protein sequence; dashes represent the absence of amino acids in the Nicotiana tabacum cv. Petite Havana SR1 protein sequences corresponding to amino acids in the Nicotiana tabacum cv. Xanthi protein sequence.

### DETAILED DESCRIPTION OF DIFFERENT EMBODIMENTS OF THE INVENTION

The current invention is based on the finding that XylT genes and XylT cDNAs from Nicotiana species and cultivars, particularly Nicotiana benthamiana and Nicotiana tabacum cv. Petite Havana SR1, are excellent source nucleotide sequences to obtain plants of those Nicotiana species and cultivars, particularly Nicotiana benthamiana plants and Nicotiana tabacum cv. Petite Havana SR1 plants, respectively, having a low level of beta-1,2-xylose residues on protein-bound N-glycans, e.g., by modifying the activity of endogenous Nicotiana XylT gene(s), by exchanging an endogenous Nicotiana XylT gene for another allele of the Nicotiana XylT gene which provides a low level of beta-1,2-xylose residues on protein-bound N-glycans, or by any combination thereof.

In one embodiment, the invention is related to a method for obtaining a Nicotiana plant cell or plant having a low level of beta-1,2-xylose residues on protein-bound N-glycans by reducing the expression of the endogenous XylT gene(s) in the Nicotiana plant cell or plant by providing one or more silencing RNA molecules to plant cells or plants of a Nicotiana species or cultivar, wherein the silencing RNA molecules comprise a part of a nucleotide sequence encoding a Nicotiana XylT protein, preferably obtained from said Nicotiana species or cultivar, wherein said part encodes at least one Nicotiana species- or cultivar-specific XylT amino acid, or wherein the silencing RNA molecules comprise a part of a nucleotide sequence of a Nicotiana XylT gene or a Nicotiana XylT cDNA, preferably obtained from said Nicotiana species or cultivar, wherein said part comprises at least one Nicotiana species- or cultivar-specific XylT nucleotide.

As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having 95% sequence identity to the sequence of the target nucleic acid. Other silencing RNA may be “unpolyadenylated RNA” comprising at least 20 consecutive nucleotides having 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other). The sense and antisense region may also be present within one RNA molecule such that a hairpin RNA (hpRNA) can be formed when the sense and antisense region form a double stranded RNA region hpRNA is well-known within the art (see e.g WO99/53050, herein incorporated by reference). The hpRNA may be classified as long hpRNA, having long, sense and antisense regions which can be largely complementary, but need not be entirely complementary (typically larger than about 200 bp, ranging between 200-1000 bp). hpRNA can also be rather small ranging in size from about 30 to about 42 bp, but not much longer than 94 bp (see WO04/073390, herein incorporated by reference). Silencing RNA may also be artificial micro-RNA molecules as described e.g. in WO2005/052170, WO2005/047505 or US 2005/0144667 (all documents incorporated herein by reference)

In another embodiment, the silencing RNA molecules are provided to the plant cell or plant of the Nicotiana species or cultivar by producing a transgenic plant cell or plant of the Nicotiana species or cultivar comprising a chimeric gene capable of producing a silencing RNA molecule, particularly a double stranded RNA (“dsRNA”) molecule, wherein the complementary RNA strands of such a dsRNA molecule comprises a part of a nucleotide sequence encoding a Nicotiana XylT protein, preferably obtained from said Nicotiana species or cultivar, wherein said part encodes at least one Nicotiana species- or cultivar-specific XylT amino acid, or wherein the complementary RNA strands of such a dsRNA molecule comprises a part of the nucleotide sequence of a Nicotiana XylT gene or a Nicotiana XylT cDNA, preferably obtained from said Nicotiana species or cultivar, wherein said part comprises at least one Nicotiana species- or cultivar-specific XylT nucleotide.

“Nicotiana”, as used herein, includes all known Nicotiana species, such as, but not limited to, Nicotiana acaulis, N. acuminata, N. africana, N. alata, N. amplexicaulis, N. arentsii, N. attenuata, N. benavidesii, N. benthamiana, N. bigelovii, N. bonariensis, N. cavicola, N. clevelandii, N. cordifolia, N. corymbosa, N. debneyi, N. excelsior, N. forgetiana, N. fragrans, N. glauca, N. glutinosa, N. goodspeedii, N. gossei, N. hybrid, N. ingulba, N. kawakamii, N. knightiana, N. langsdorffii, N. linearis, N. longiflora, N. maritima, N. megalosiphon, N. miersii, N. noctijlora, N. nudicaulis, N. obtusifolia, N. occidentalis, N. otophora, N. paniculata, N. paucijlora, N. petunioides, N. plumbaginifolia, N. quadrivalvis, N. raimondii, N. repanda, N. rosulata, N. rotundifolia, N. rustica, N. setchellii, N. simulans, N. solanifolia, N. spegazzinii, stocktonii, N. suaveolens, N. sylvestris, N. tabacum, N. thyrsijlora, N. tomentosa, N. tomentosiformis, N. trigonophylla, N. umbratica, N. undulata, N. velutina, N. wigandioides, and Nicotiana×sandera, and all known Nicotiana cultivars, such as, but not limited to, cultivars of Nicotiana tabacum, such as cv. Burley21, cv. Delgold, cv. Petit Havana, cv. Petit Havana SR1, cv. Samsun, and cv. Xanthi.

Nicotiana tabacum, which is common tobacco, is a tetraploid hybrid species, which originated from the diploid species Nicotiana sylvestris and Nicotiana tomentosiformis.

A Nicotiana XylT gene or a Nicotiana XylT cDNA refers to a nucleotide sequence of a XylT gene that naturally occurs in a Nicotiana species or cultivar or to cDNA corresponding to the mRNA of a XylT gene that naturally occurs in a Nicotiana species or cultivar. Similarly, a Nicotiana XylT protein refers to a protein as it naturally occurs in a Nicotiana species or cultivar.

Examples of nucleotide sequences encoding a Nicotiana XylT protein, include those obtained from Nicotiana benthamiana encoding the amino acid sequence set forth in SEQ ID No.: 12 or SEQ ID No.: 14, and those obtained from Nicotiana tabacum cv. Petite Havana SR1 encoding the amino acid sequence set forth in SEQ ID No.: 4, SEQ ID No.:6, SEQ ID No.: 8, or SEQ ID No.:10.

Examples of nucleotide sequences of a Nicotiana XylT gene include those obtained from Nicotiana benthamiana comprising the nucleotide sequence set forth in SEQ ID No.: 11, SEQ ID No.: 13, or SEQ ID No.: 21, and those obtained from Nicotiana tabacum cv. Petite Havana SR1 comprising the nucleotide sequence set forth in SEQ ID No.: 7 or SEQ ID No.: 9.

Examples of nucleotide sequences of a Nicotiana XylT cDNA, include those obtained from Nicotiana tabacum cv. Petite Havana SR1 comprising the nucleotide sequence set forth in SEQ ID No.: 3, SEQ ID No.:5 or SEQ ID No.: 17.

However, it will be immediately clear to the person skilled in the art that the exemplified nucleotide sequences or parts thereof can be used to identify further nucleotide sequences of Nicotiana XylT genes or Nicotiana XylT cDNAs in Nicotiana species or cultivars, and that such nucleotide sequences or parts thereof may also be used e.g. to decrease the level of beta-1,2-xylose residues on protein-bound N-glycans in Nicotiana plants. The exemplified nucleotide sequences could be used to select:
i) a DNA fragment comprising a nucleotide sequence encoding the amino acid sequence of SEQ ID No.: 4, SEQ ID No.:6, SEQ ID No.: 8, SEQ ID No.:10, SEQ ID No.: 12, or SEQ ID No.:14, for use as a probe;
ii) a DNA fragment comprising the nucleotide sequence of any one of SEQ ID No.; 3, SEQ ID No.: 5, SEQ ID No.: 7, SEQ ID No.: 9, SEQ ID No.: 11, SEQ ID No.: 13, SEQ ID No.: 17, or SEQ ID No.: 21, for use as a probe;
iii) a DNA fragment or oligonucleotide comprising a nucleotide sequence consisting of between 20 to 1513 consecutive nucleotides selected from a nucleotide sequence encoding the amino acid sequence of SEQ ID No.: 4, or SEQ ID No.:6, for use as a probe;
iv) a DNA fragment or oligonucleotide comprising a nucleotide sequence consisting of between 20 to 3574 consecutive nucleotides selected from a nucleotide sequence encoding the amino acid sequence of SEQ ID No.: 8, SEQ ID No.:10, SEQ ID No.: 12, or SEQ ID No.:14, for use as a probe
v) a DNA fragment or oligonucleotide comprising a nucleotide sequence consisting of between 20 to 3574 consecutive nucleotides selected from a nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21, for use as a probe;
vi) an oligonucleotide sequence having a nucleotide sequence comprising between 20 to 200 consecutive nucleotides selected from a nucleotide sequence encoding the amino acid sequence of SEQ ID No.; 4, or SEQ ID No.:6, for use as a primer in a PCR reaction;
vii) an oligonucleotide sequence having a nucleotide sequence comprising between 20 to 200 consecutive nucleotides selected from a nucleotide sequence encoding the amino acid sequence of SEQ ID No.: 8, SEQ ID No.:10, SEQ ID No.: 12, or SEQ ID No.:14, for use as a primer in a PCR reaction;
viii) an oligonucleotide sequence having a nucleotide sequence comprising between 20 to 200 consecutive nucleotides selected from the nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21, for use as a primer in a PCR reaction; or
ix) an oligonucleotide having the nucleotide sequence of any one of SEQ ID No.: 1, SEQ ID No.: 2, SEQ ID No.: 15 or SEQ ID No.: 16, SEQ ID No.:19 or SEQ ID No.20 for use as a primer in a PCR reaction.

By performing a PCR using genomic DNA or cDNA from Nicotiana species or cultivars and the mentioned oligonucleotides as primers or by performing hybridization, preferably under stringent conditions between genomic or cDNA from Nicotiana species or cultivars and the mentioned probes, such other Nicotiana XylT genes or Nicotiana XylT cDNAs or fragments thereof can be identified and/or isolated.

“Stringent hybridization conditions” as used herein means that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C., e.g. for about 10 min (twice). Other hybridization and wash conditions are well known and are exemplified in Sambrook et al, Molecular Cloning: A Laboratory Manual, Second Edition, Cold Spring Harbor, N.Y. (1989), particularly chapter 11.

A “Nicotiana species-specific XylT nucleotide” or a “Nicotiana cultivar-specific XylT nucleotide”, refers to a nucleotide of the nucleotide sequence of a XylT gene or a XylT cDNA from a Nicotiana species or cultivar that differs from or is not present in the corresponding nucleotide sequence of the XylT gene from Nicotiana tabacum cv. Xanthi represented in SEQ ID NO: 25, or of the XylT cDNA from Nicotiana tabacum cv. Xanthi represented in SEQ ID NO: 23, respectively.

A “Nicotiana species-specific XylT amino acid” or a “Nicotiana cultivar-specific XylT amino acid”, refers to an amino acid of the amino acid sequence of a XylT protein encoded by a XylT gene or encoded by a XylT cDNA from a Nicotiana species or cultivar that differs from or is not present in the corresponding amino acid sequence of the XylT protein encoded by the XylT gene from Nicotiana tabacum cv. Xanthi represented in SEQ ID NO: 26, or encoded by the XylT cDNA from Nicotiana tabacum cv. Xanthi represented in SEQ ID NO: 24, respectively.

To determine the presence of a Nicotiana species- or cultivar-specific XylT nucleotide or amino acid in the nucleotide sequence of a XylT gene or a XylT cDNA from a Nicotiana species or cultivar or in the amino acid sequence of a XylT protein encoded by a XylT gene or encoded by a XylT cDNA from a Nicotiana species or cultivar, for the purpose of this invention, the XylT nucleotide or amino acid sequences from the Nicotiana species or cultivar are compared with the corresponding XylT nucleotide or amino acid sequences from Nicotiana tabacum cv. Xanthi by aligning the sequences using a global alignment procedure (For nucleotide sequences the default scoring matrix used is “standard linear” with mismatch penalty=2, open gap penalty=4 and extend gap penalty=1. For protein sequences the default scoring matrix is “blosum 62”; Henikoff and Henikoff, 1992.). To perform the alignment the Align Plus program (provided by Scientific & Educational Software, USA) may be used.

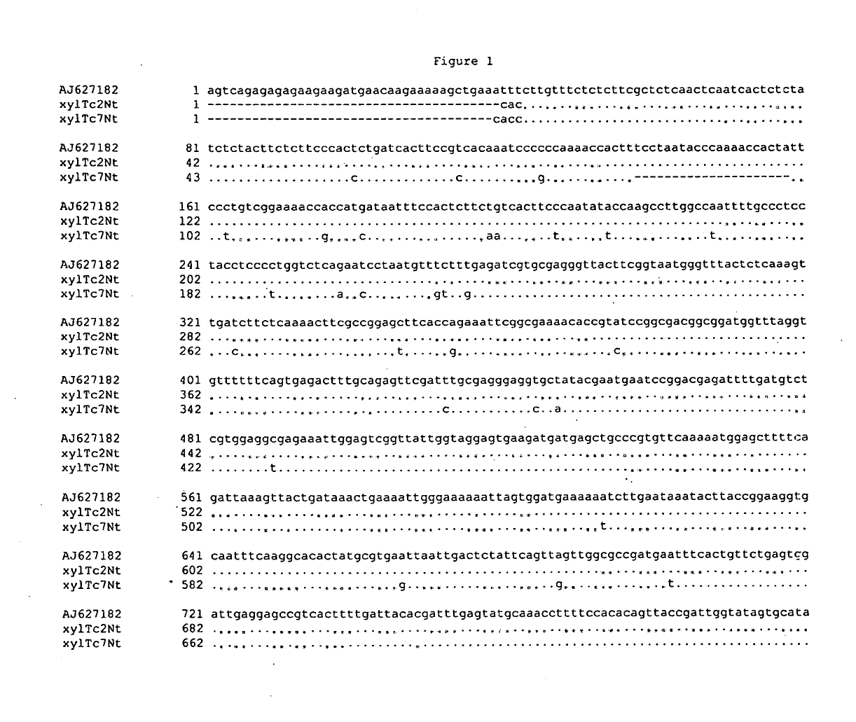

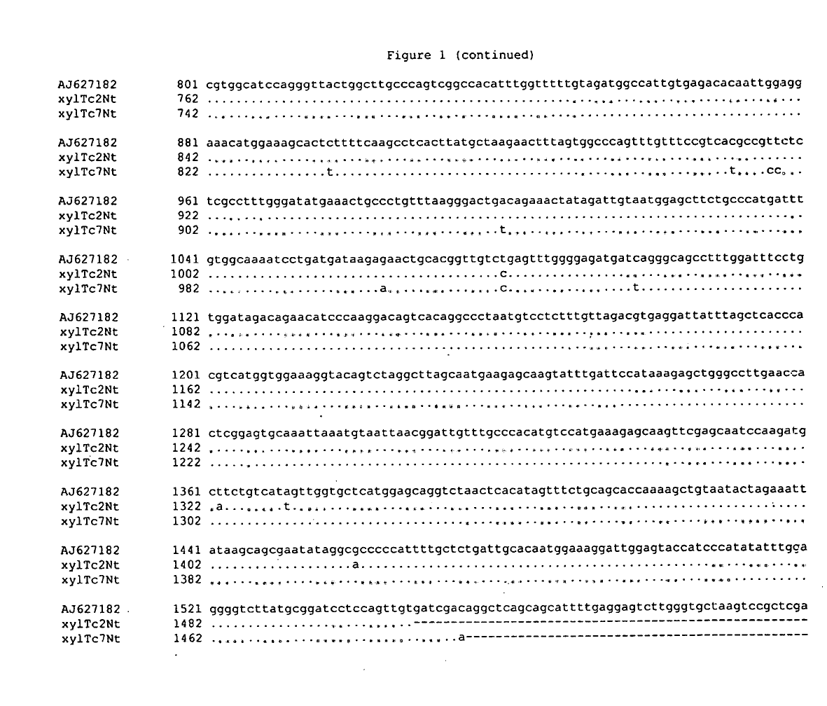

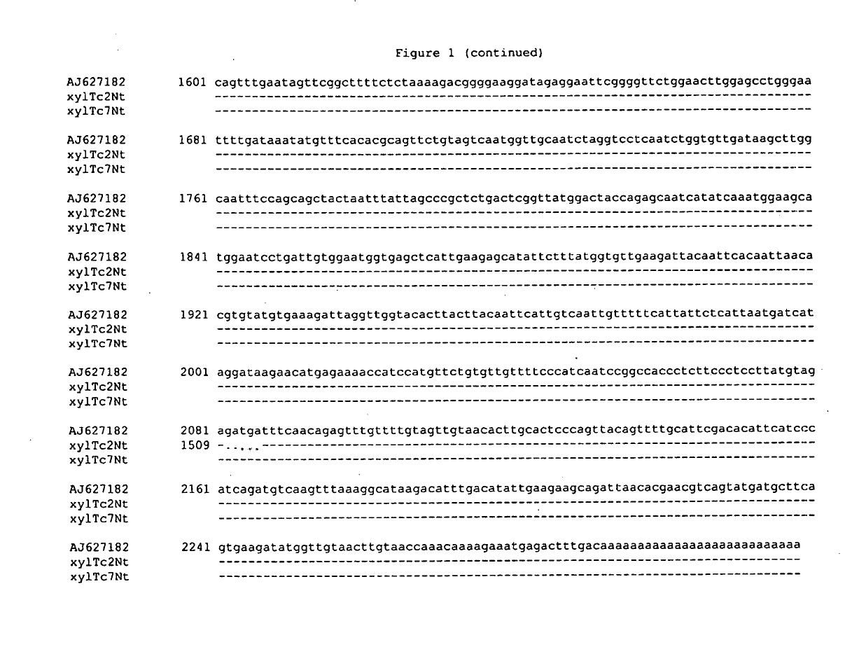

One example of such a global DNA alignment is the global DNA alignment of the XylT cDNA sequence from Nicotiana tabacum cv. Xanthi represented in SEQ ID NO:23 with the XylT cDNA sequences from Nicotiana tabacum cv. Petite Havana SR1 represented in SEQ ID NO:3 and 5, in FIG. 1. Examples of Nicotiana tabacum cv. Petite Havana SR1-specific XylT nucleotides determined based on this global DNA alignment include:
the nucleotide at position 1041, 1323, 1332, or 1421 of SEQ ID NO:3,
the nucleotide at position 62, 76, 87, 104, 117, 122, 139, 140, 148, 155, 169, 190, 199, 202, 212, 213, 216, 265, 287, 294, 316, 373, 385, 388, 430, 554, 607, 628, 643, 838, 892, 897, 898, 941, 1005, 1021, 1039, or 1495 of SEQ ID NO:5.

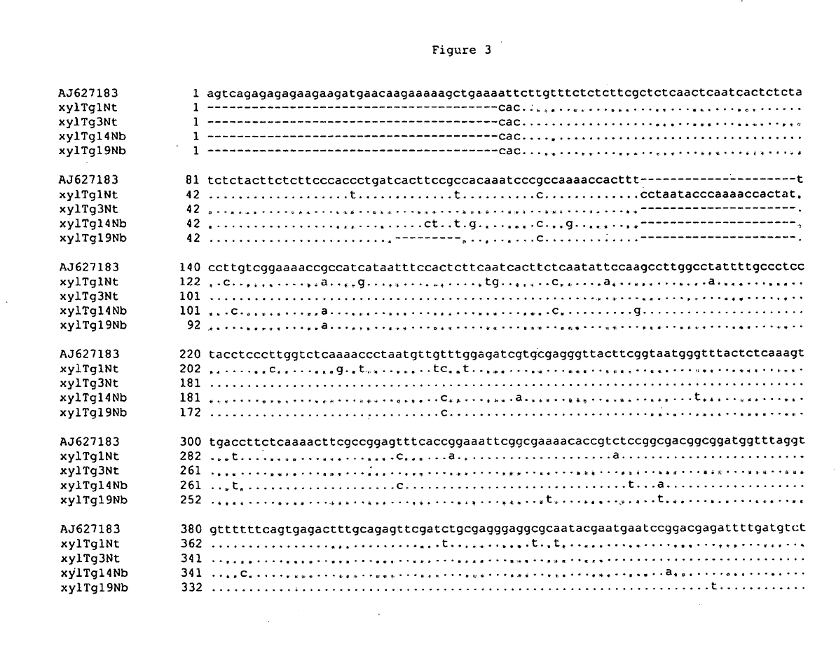

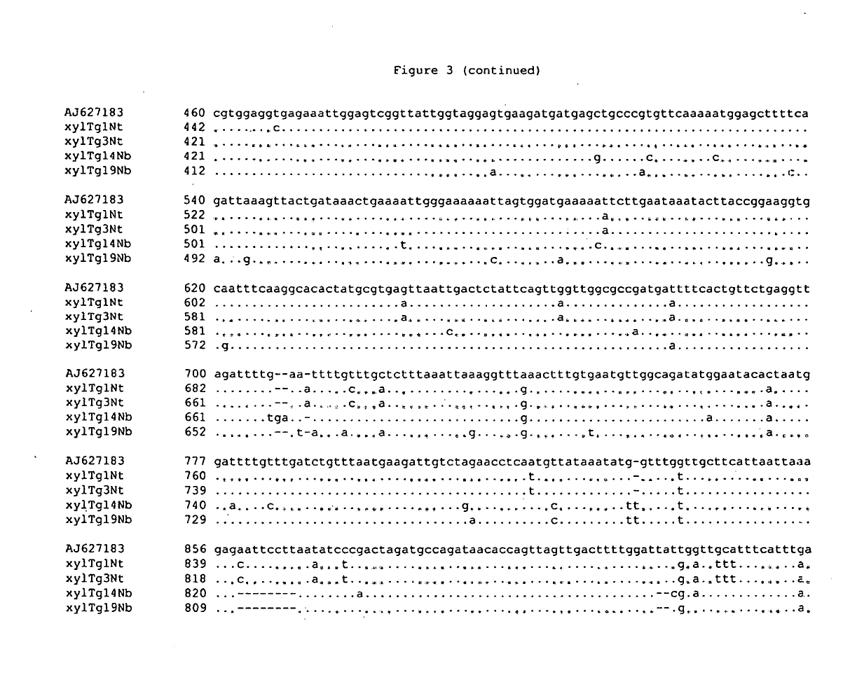

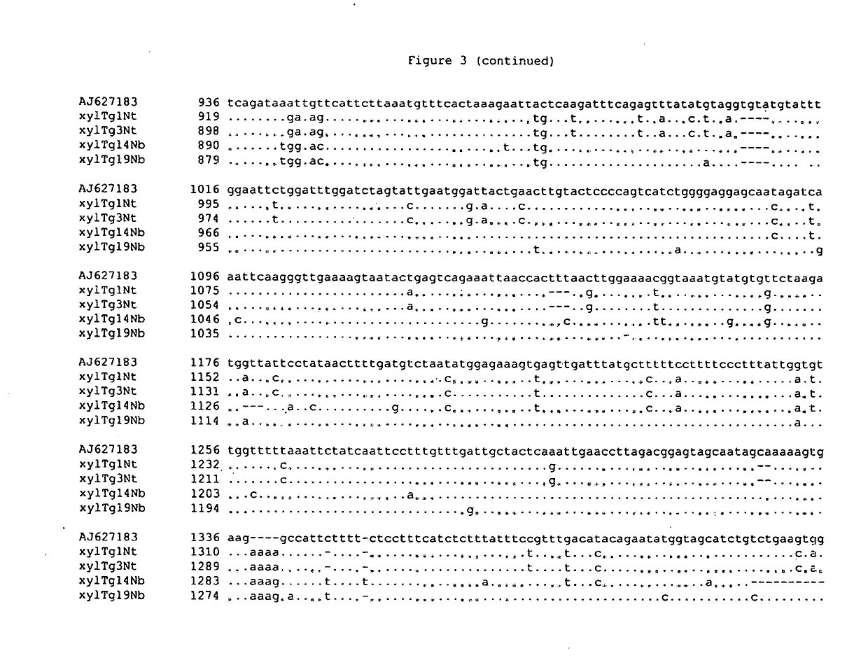

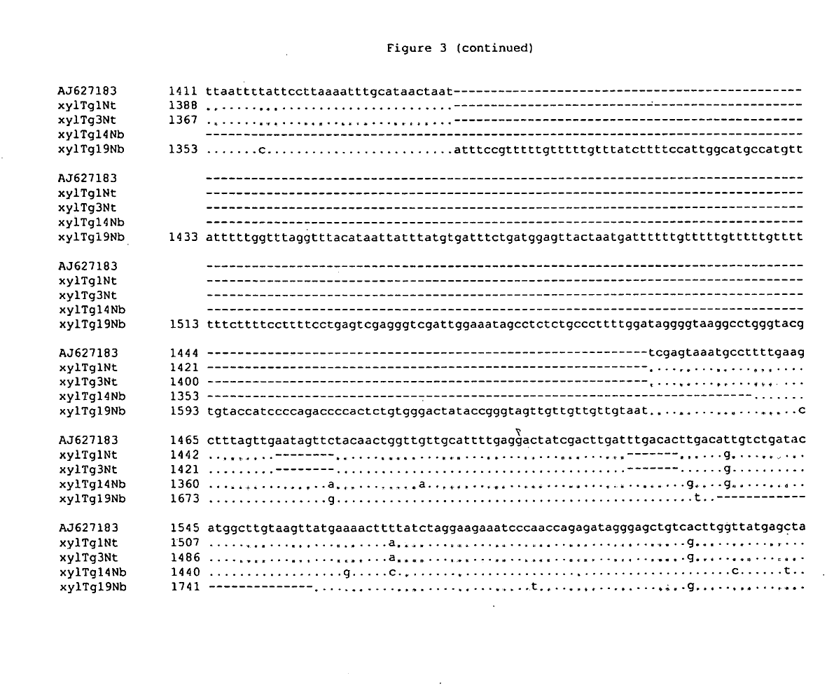

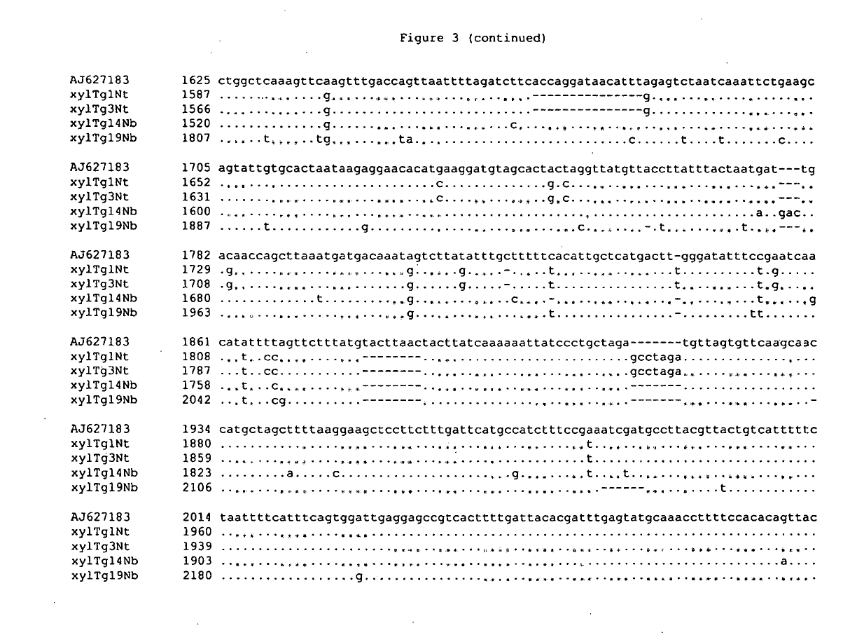

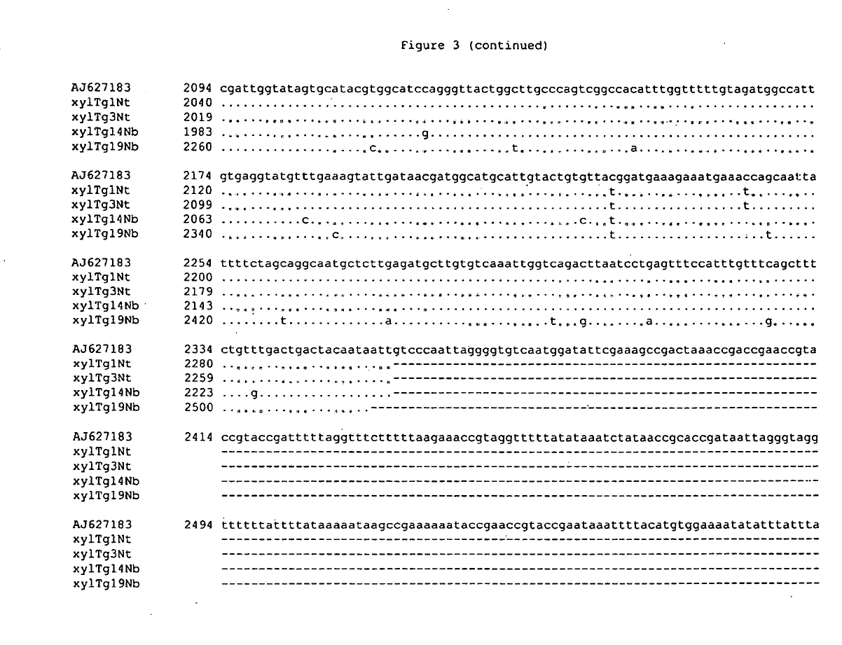

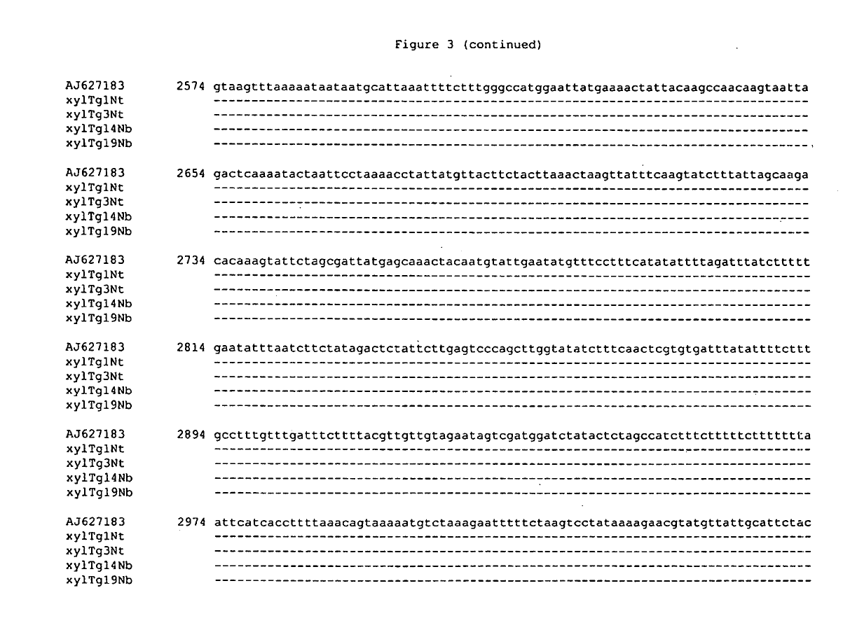

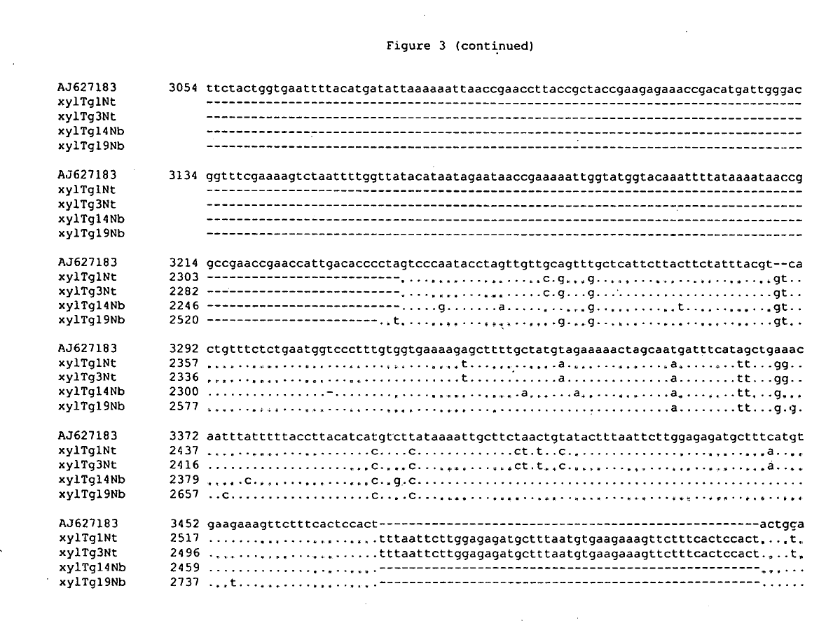

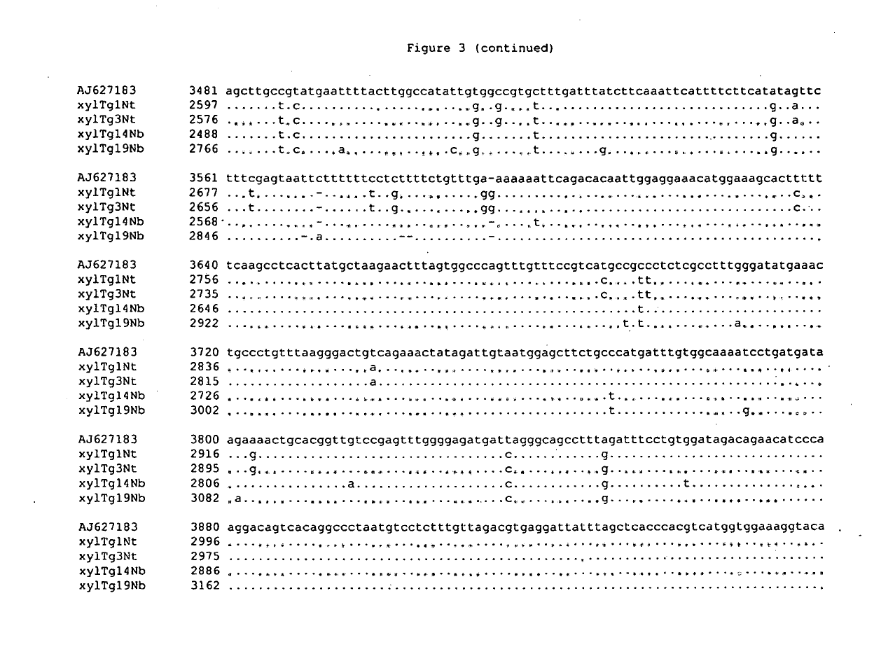

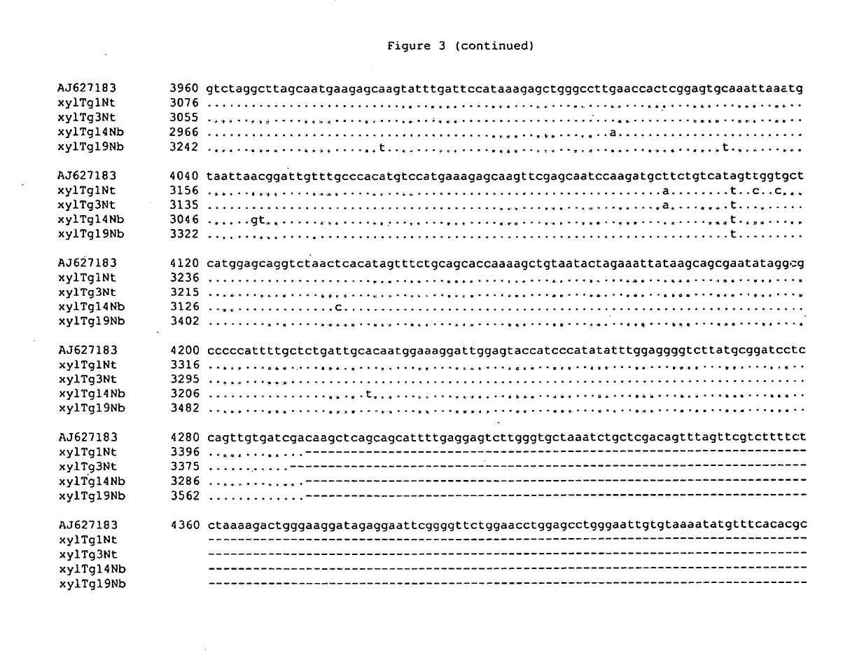

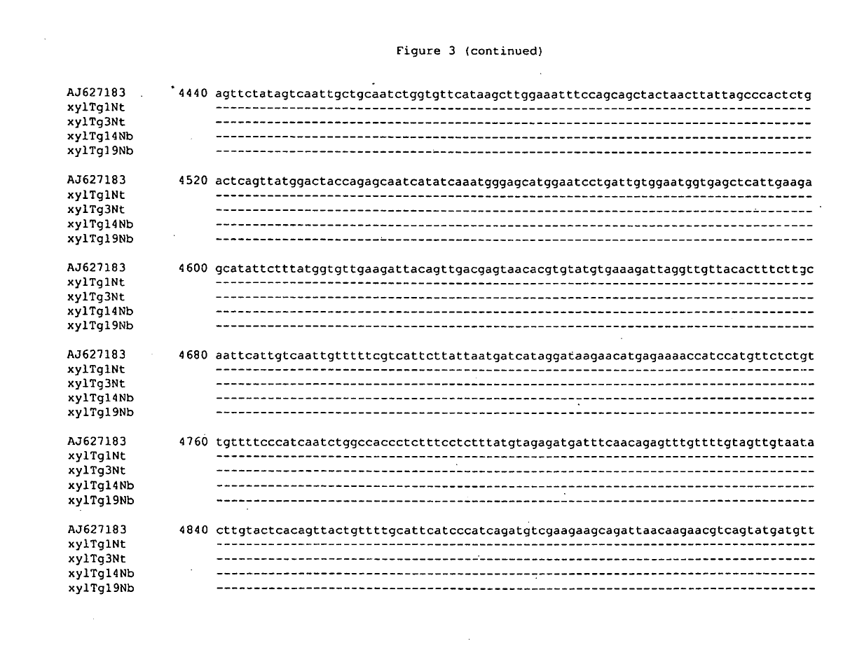

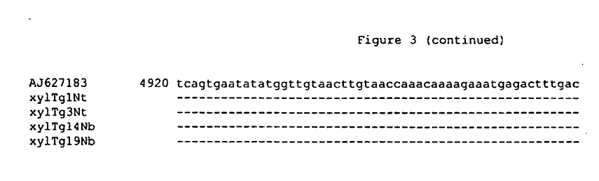

Another example of such a global DNA alignment is the global DNA alignment of the XylT gene sequence from Nicotiana tabacum cv. Xanthi represented in SEQ ID NO:25 with the XylT gene sequences from Nicotiana tabacum cv. Petite Havana SR1 represented in SEQ ID NO:7 and 9 and with the XylT gene sequences from Nicotiana benthamiana represented in SEQ ID NO:11 and 13, in FIG. 3. Examples of Nicotiana tabacum cv. Petite Havana SR1-specific XylT nucleotides determined based on this global DNA alignment include:
the nucleotide at position 61, 75, 86, 100-120, 124, 137, 142, 159, 160, 168, 175, 189, 210, 219, 222, 232, 233, 236, 285, 307, 314, 336, 393, 405, 408, 450, 574, 627, 648, 663, 692, 698, 702, 721, 754, 802, 821, 842, 852, 856, 901, 903, 906, 907, 908, 917, 927, 928, 930, 931, 960, 961, 965, 974, 977, 981, 983, 986, 1001, 1019, 1027, 1029, 1034, 1068, 1073, 1099, 1120, 1129, 1144, 1154, 1158, 1181, 1193, 1208, 1212, 1228, 1230, 1239, 1275, 1313-1316, 1348, 1353, 1357, 1384, 1386, 1496, 1531, 1571, 1601, 1629, 1681, 1696, 1698, 1730, 1754, 1761, 1772, 1789, 1800, 1802, 1811, 1814, 1815, 1855-1861, 1929, 2172, 2190, 2322, 2324, 2328, 2353, 2354, 2391, 2404, 2419, 2428, 2429, 2433, 2434, 2459, 2464, 2478, 2479, 2481, 2484, 2512, 2540-2590, 2595, 2604, 2606, 2630, 2633, 2638, 2670, 2673, 2680, 2695, 2698, 2710, 2711, 2752, 2806, 2811, 2812, 2855, 2919, 2953, 2966, 3217, 3226, 3229, or 3232 of SEQ ID NO:7,
the nucleotide at position 553, 606, 627, 642, 671, 677, 681, 700, 733, 781, 800, 821, 831, 835, 880, 882, 885, 886, 887, 896, 906, 907, 909, 910, 939, 940, 944, 953, 956, 960, 962, 965, 980, 998, 1006, 1008, 1013, 1047, 1052, 1078, 1099, 1108, 1123, 1133, 1137, 1160, 1172, 1187, 1191, 1207, 1209, 1218, 1254, 1292, 1293, 1294, 1295, 1327, 1332, 1336, 1363, 1365, 1475, 1510, 1550, 1580, 1608, 1660, 1675, 1677, 1709, 1733, 1740, 1751, 1768, 1779, 1781, 1790, 1793, 1794, 1834-1840, 1908, 2151, 2169, 2301, 2303, 2307, 2332, 2333, 2370, 2383, 2398, 2407, 2408, 2412, 2413, 2438, 2443, 2457, 2458, 2460, 2463, 2491, 2519-2569, 2574, 2583, 2585, 2609, 2612, 2617, 2649, 2652, 2659, 2674, 2677, 2689, 2690, 2731, 2785, 2790, 2791, 2834, 2898, 2932, 2945, 3196, or 3205 of SEQ ID NO:9

Examples of Nicotiana benthamiana-specific XylT nucleotides determined based on this global DNA alignment include:
the nucleotide at position 71, 72, 75, 77, 86, 90, 104, 116, 147, 158, 212, 222, 246, 264, 286, 317, 321, 345, 402, 472, 479, 488, 526, 552, 612, 637, 668, 669, 670, 701, 726, 734, 742, 747, 773, 785, 795, 796, 802, 831, 871, 872, 874, 888, 897, 898, 899, 901, 902, 927, 931, 932, 1039, 1044, 1047, 1080, 1091, 1103, 1104, 1113, 1118, 1131, 1134, 1145, 1152, 1164, 1179, 1183, 1199, 1201, 1206, 1227, 1286, 1287, 1288, 1289, 1296, 1301, 1317, 1328, 1332, 1347, 1376, 1388, 1424, 1429, 1458, 1464, 1510, 1517, 1534, 1559, 1672, 1675, 1676, 1677, 1693, 1705, 1719, 1750, 1757, 1761, 1765, 1832, 1838, 1862, 1872, 1877, 1978, 2010, 2074, 2111, 2115, 2227, 2251, 2259, 2271, 2283, 2296, 2297, 2341, 2348, 2361, 2370, 2371, 2375, 2384, 2401, 2404, 2406, 2495, 2497, 2521, 2529, 2561, 2607, 2701, 2777, 2822, 2843, 2856, 2867, 3020, 3052, 3053, 3116, 3143, or 3227 of SEQ ID NO:11
the nucleotide at position 77, 107, 203, 297, 312, 399, 449, 469, 489, 492, 496, 529, 538, 566, 573, 633, 661, 662, 666, 671, 683, 690, 699, 723, 763, 774, 784, 785, 791, 861, 877, 886, 887, 888, 890, 891, 920, 921, 943, 996, 1015, 1034, 1116, 1190, 1226, 1277-1280, 1282, 1287, 1331, 1343, 1360, 1386-1651, 1672, 1689, 1738, 1770, 1791, 1813, 1820, 1822, 1831, 1832, 1862, 1869, 1874, 1882, 1893, 1906, 1935, 1945, 1956, 1988, 2007, 2033, 2034, 2045, 2049, 2050, 2167, 2198, 2280, 2299, 2315, 2355, 2392, 2413, 2428, 2442, 2464, 2468, 2477, 2493, 2522, 2544, 2548, 2573, 2574, 2639, 2648, 2649, 2653, 2655, 2659, 2679, 2684, 2740, 2773, 2775, 2781, 2796, 2799, 2807, 2816, 2839, 2857, 2975, 2977, 2990, 3053, 3071, 3083, 3119, 3132, 3265, 3311, or 3392 of SEQ ID NO:13.

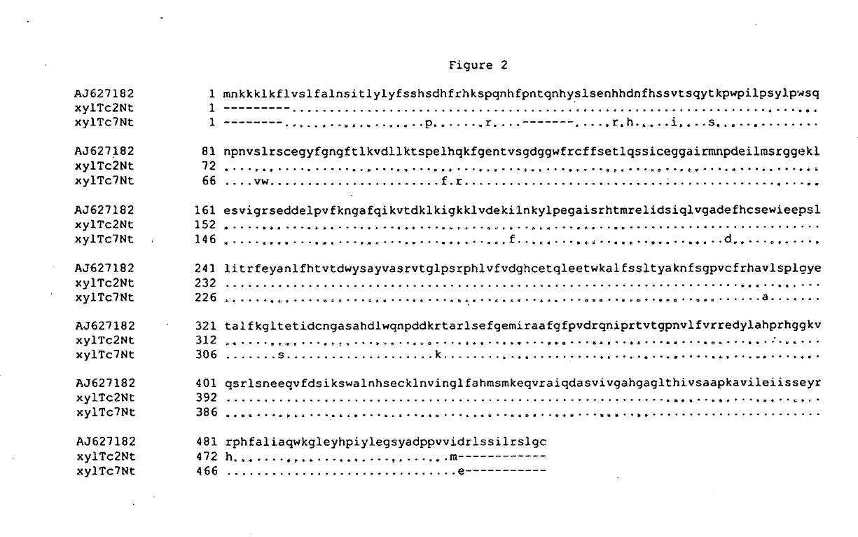

One example of such a global protein alignment is the global protein alignment of the XylT protein sequence encoded by the XylT cDNA sequence from Nicotiana tabacum cv. Xanthi represented in SEQ ID NO:24 with the XylT protein sequences encoded by the XylT cDNA sequences from Nicotiana tabacum cv. Petite Havana SR1 represented in SEQ ID NO:4 and 6, in FIG. 2. Examples of Nicotiana tabacum cv. Petite Havana SR1-specific XylT amino acids determined based on this global protein alignment include:
the amino acid at position 472 or 502 of SEQ ID NO:4,
the amino acid at position 20, 28, 38, 40, 46, 51, 70, 71, 95, 97, 184, 213, 298, 313, 334, or 497 of SEQ ID NO:6.

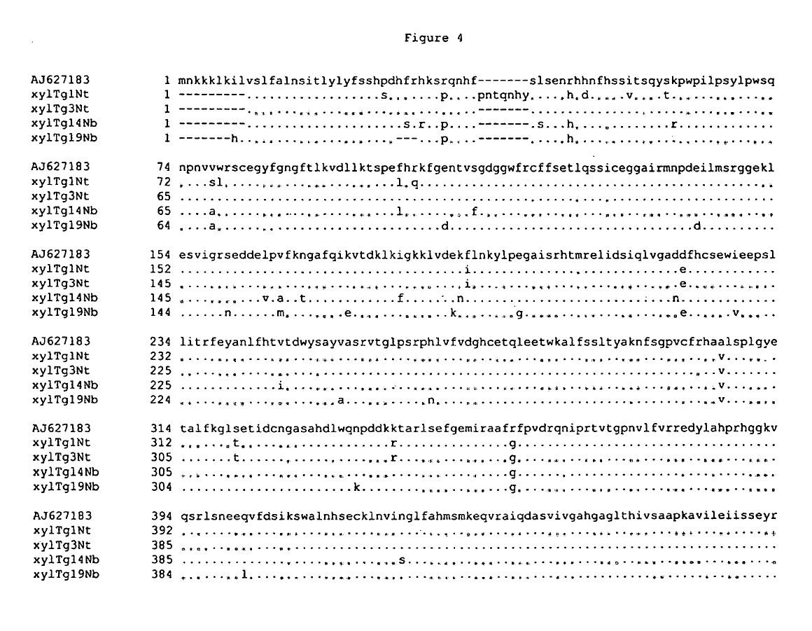

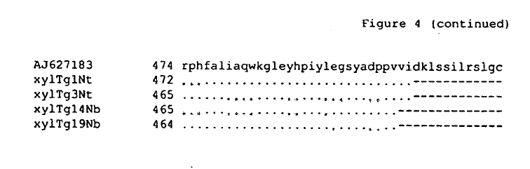

Another example of such a global protein alignment is the global protein alignment of the XylT protein sequences encoded by the XylT gene sequence from Nicotiana tabacum cv. Xanthi represented in SEQ ID NO:26 with the XylT protein sequences encoded by the XylT gene sequences from Nicotiana tabacum cv. Petite Havana SR1 represented in SEQ ID NO:8 and 10 and with the XylT protein sequences encoded by the XylT gene sequences from Nicotiana benthamiana represented in SEQ ID NO:12 and 14, in FIG. 4. Examples of Nicotiana tabacum cv. Petite Havana SR1-specific XylT amino acids determined based on this global protein alignment include:
the amino acid at position 19, 27, 32-38, 44, 46, 52, 57, 76, 77, 101, 103, 190, 219, 304, 319, 340, or 356 of SEQ ID NO:8,
the amino acid at position 183, 212, 297, 312, 333, or 349 of SEQ ID NO:10.

Examples of Nicotiana benthamiana-specific XylT amino acids determined based on this global protein alignment include:
the amino acid at position 22, 24, 27, 33, 37, 51, 69, 94, 104, 156, 158, 161, 174, 182, 211, 238, 297, 349, or 414 of SEQ ID NO:12,
the amino acid at position 1, 26, 36, 68, 99, 133, 150, 157, 166, 180, 189, 211, 218, 245, 257, 296, 327, 348, or 392 of SEQ ID NO:14.

The part of the nucleotide sequence encoding a Nicotiana XylT protein and the part of the nucleotide sequence of a Nicotiana XylT gene or a Nicotiana XylT cDNA comprised within the silencing RNA molecule, particularly within one strand of the double stranded RNA molecule, should be at least 19 nucleotides long, but may vary from about 19 nucleotides (nt) up to a length equaling the length (in nucleotides) of the Nicotiana XylT protein-encoding sequence or the Nicotiana XylT gene or cDNA sequence. The total length of the sense or antisense nucleotide sequence may thus be at least 25 nt, or at least about 50 nt, or at least about 100 nt, or at least about 150 nt, or at least about 200 nt, or at least about 500 nt. It is expected that there is no upper limit to the total length of the sense or the antisense nucleotide sequence. However for practical reason (such as e.g. stability of the chimeric genes) it is expected that the length of the sense or antisense nucleotide sequence should not exceed 5000 nt, particularly should not exceed 2500 nt and could be limited to about 1000 nt.

It will be appreciated that the longer the total length of the part of nucleotide sequence encoding a Nicotiana XylT protein or the part of the nucleotide sequence of a Nicotiana XylT gene or a Nicotiana XylT cDNA (sense or antisense region), the less stringent the requirements for sequence identity between these regions and the corresponding sequence in the endogenous XylT gene from the Nicotiana species or cultivar it complements are. Preferably, the nucleic acid of interest should have a sequence identity of at least about 75% with the corresponding target sequence, particularly at least about 80%, more particularly at least about 85%, quite particularly about 90%, especially. about 95%, more especially about 100%, quite especially be identical to the corresponding part of the target sequence or its complement. However, it is preferred that the nucleic acid of interest always includes a sequence of about 19 consecutive nucleotides, particularly about 25 nt, more particularly about 50 nt, especially about 100 nt, quite especially about 150 nt with 100% sequence identity to the corresponding part of the target XylT nucleic acid, wherein said about 19 consecutive nucleotides, particularly about 25 nt, more particularly about 50 nt, especially about 100 nt, quite especially about 150 nt, encode at least one Nicotiana species- or cultivar-specific XylT amino acid or comprise at least one Nicotiana species- or cultivar-specific XylT nucleotide.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. Preferably, for calculating the sequence identity and designing the corresponding sense or antisense sequence, the number of gaps should be minimized, particularly for the shorter sense sequences.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

It has been demonstrated that the minimum requirement for silencing a particular target gene is the presence in the silencing chimeric gene nucleotide sequence of a nucleotide sequence of about 20-21 consecutive nucleotides long corresponding to the target gene sequence, in which at least 19 of the 20-21 consecutive nucleotides are identical to the corresponding target gene sequence. “19 out of 20 consecutive nucleotides” as used herein refers to a nucleotide sequence of 20 consecutive nucleotides selected from the target gene having one mismatch nucleotide.

For silencing the endogenous XylT gene from a Nicotiana species or cultivar, it is preferred that the silencing chimeric gene nucleotide sequence comprises at least 19 out of 20-21 consecutive nucleotides from a nucleotide sequence encoding a Nicotiana XylT protein, wherein said at least 19 out of 20-21 consecutive nucleotides encode at least one Nicotiana species- or cultivar-specific XylT amino acid, or comprises at least 19 out of 20-21 consecutive nucleotides from a nucleotide sequence of a Nicotiana XylT gene or a Nicotiana XylT cDNA, wherein said at least 19 out of 20-21 consecutive nucleotides comprise at least one Nicotiana species- or cultivar-specific XylT nucleotide.

As used herein “a Nicotiana plant having a low level of beta-1,2-xylose residues on protein-bound N-glycans” is a plant (particularly a Nicotiana plant obtained according to the methods of the invention), in which the XylT activity is decreased or abolished resulting in a lower level of beta-1,2-xylose residues on protein-bound N-glycans than the level of beta-1,2-xylose residues on protein-bound N-glycans in a control Nicotiana plant not treated according to the methods of the invention or resulting in the absence of beta-1,2-xylose residues on protein-bound N-glycans. An indication of XylT activity can be obtained by comparing the level of beta-1,2-xylose residues present on the glycans of proteins from the Nicotiana plant obtained according to the methods of the invention with the level of beta-1,2-xylose residues present on the glycans of proteins from a control Nicotiana plant not treated according to the methods of the invention. The level of beta-1,2-xylose residues on protein-bound N-glycans of plants can be measured e.g. by Western blot analysis using xylose-specific antibodies as described e.g. by Faye et al. (Analytical Biochemistry, 1993, 209: 104-108) or by mass spectrometry on glycans isolated from the plant's glycoproteins using Matrix-assisted Laser Desorption/Ionization Time-of-Flight mass spectronomy (MALDI-TOF-MS) as described e.g. by Kolarich and Altmann (2000, Anal. Biochem. 285: 64-75) or using Liquid Chromatography Tandem mass spectronomy (LC/MS/MS) as described e.g. by Henriksson et al. (2003, Biochem. J. 375: 61-73).

dsRNA encoding Nicotiana XylT expression reducing chimeric genes according to the invention may comprise an intron, such as a heterologous intron, located e.g. in the spacer sequence between the sense and antisense RNA regions in accordance with the disclosure of WO 99/53050 (incorporated herein by reference).

It has become apparent that double stranded RNA molecules, such as the ones described above, are cleaved in plant cells into small RNA fragments of about 20-21 nucleotides, which serve as guide sequence in the degeneration of the corresponding mRNA (reviewed by Baulcombe, 2004). Thus, in another embodiment, the invention is drawn to a method for producing a Nicotiana plant cell or plant having a low level of beta-1,2-xylose residues on protein-bound N-glycans comprising the steps of
a) providing one or more double stranded RNA molecules to cells of a plant of a Nicotiana species or cultivar, wherein the double stranded RNA molecules comprise two RNA strands, one RNA strand consisting essentially of an RNA nucleotide sequence of 20 to 21 consecutive nucleotides selected from a nucleotide sequence encoding a Nicotiana XylT protein, preferably obtained from said Nicotiana species or cultivar, wherein said 20 to 21 consecutive nucleotides encode at least one Nicotiana species- or cultivar-specific XylT amino acid, or one RNA strand consisting essentially of an RNA nucleotide sequence of 20 to 21 consecutive nucleotides from a nucleotide sequence of a Nicotiana XylT gene or a Nicotiana XylT cDNA, preferably obtained from said Nicotiana species or cultivar, wherein said 20 to 21 consecutive nucleotides comprise at least one Nicotiana species- or cultivar-specific XylT nucleotide; and
b) identifying a Nicotiana plant comprising these double stranded RNA molecule or molecules which has a lower level of beta-1,2-xylose residues on protein-bound N-glycans than a same Nicotiana plant which does not comprise the double stranded RNA molecule or molecules.

The mentioned 20-21 nt long dsRNA sequences are also generated in the course of conventional antisense RNA mediated silencing or sense RNA mediated silencing. Thus, in another embodiment of the invention, a method is provided for producing a Nicotiana plant cell or plant having a low level of beta-1,2-xylose residues on protein-bound N-glycans, comprising the step of providing to cells of a plant of the Nicotiana species or cultivar a chimeric gene comprising, operably linked, the following DNA fragments
a) a plant expressible promoter;
b) a DNA region comprising at least 20 consecutive nucleotides selected from a nucleotide sequence encoding a Nicotiana XylT protein, preferably obtained from said Nicotiana species or cultivar, wherein said at least 20 consecutive nucleotides encode at least one Nicotiana species- or cultivar-specific XylT amino acid, or comprising at least 20 consecutive nucleotides from a nucleotide sequence of a Nicotiana XylT gene or a Nicotiana XylT cDNA, preferably obtained from said Nicotiana species or cultivar, wherein said at least 20 consecutive nucleotides comprise at least one Nicotiana species- or cultivar-specific XylT nucleotide, in antisense or in sense orientation;
c) a DNA region comprising a transcription termination and polyadenylation signal functional in plants.

The mentioned antisense or sense nucleotide regions may thus be from about 21 nt to about 5000 nt long, such as 21 nt, 40 nt, 50 nt, 100 nt, 200 nt, 300 nt, 500 nt, 1000 nt, or even about 2000 nt or larger in length. Moreover, it is not required for the purpose of the invention that the nucleotide sequence of the used inhibitory XylT gene molecule or the encoding region of the chimeric gene, is completely identical or complementary to the endogenous Nicotiana XylT gene the expression of which is targeted to be reduced in the Nicotiana plant cell. The longer the sequence, the less stringent the requirement for the overall sequence identity is. Thus, the sense or antisense regions may have an overall sequence identity of about 40% or 50% or 60% or 70% or 80% or 90% or 100% to the nucleotide sequence of the endogenous Nicotiana gene or the complement thereof. However, as mentioned, antisense or sense regions should preferably comprise a nucleotide sequence of 19-20 consecutive nucleotides having about 100% sequence identity to the nucleotide sequence of the XylT gene, wherein said 19-20 consecutive nucleotides, encode at least one Nicotiana species- or cultivar-specific XylT amino acid or comprise at least one Nicotiana species- or cultivar-specific XylT nucleotide. The stretch of about 100% sequence identity may be about 50, 75 or 100 nt.

The efficiency of the above mentioned chimeric genes which when transcribed yield antisense or sense silencing RNA may be further enhanced by inclusion of DNA elements which result in the expression of aberrant, unpolyadenylated XylT inhibitory RNA molecules. One such DNA element suitable for that purpose is a DNA region encoding a self-splicing ribozyme, as described in WO 00/01133. The efficiency may also be enhanced by providing the generated RNA molecules with nuclear localization or retention signals as described in WO 03/076619.

The exemplified XylT nucleotide sequences from Nicotiana benthamiana and from Nicotiana tabacum can also be used to identify XylT alleles in a population of plants of a Nicotiana species or cultivar which are correlated with low levels of beta-1,2-xylose residues on protein-bound N-glycans. Such populations of plants of a Nicotiana species or cultivar may be populations which have been previously mutagenized. The identified XylT alleles may then be introduced into a plant line of a Nicotiana species or cultivar of choice using conventional breeding techniques.

Methods to transform Nicotiana plants are also well known in the art. Agrobacterium-mediated transformation of Nicotiana has been described e.g. in Zambryski et al. (1983, EMBO J. 2: 2143-2150), De Block et al. (1984, EMBO J. 3(8):1681-1689), or Horsch et al. (Science (1985) 227: 1229-1231).

The obtained transformed Nicotiana plants according to the invention, or the obtained Nicotiana plants having a low level of beta-1,2-xylose residues on protein-bound N-glycans wherein the endogenous XylT gene has been replaced by a XylT allele, which is correlated with a lower levels of beta-1,2-xylose residues on protein-bound N-glycans than the original XylT allele, can be used in a conventional breeding scheme to produce more plants with the same characteristics or to introduce the chimeric gene according to the invention in other cultivars of the same or related plant species, or in hybrid plants. Seeds obtained from the transformed plants contain the chimeric genes of the invention as a stable genomic insert and are also encompassed by the invention.

Furthermore, it is known that introduction of antisense, sense or doublestranded RNA or the encoding chimeric genes may lead to a distribution of phenotypes, ranging from almost no or very little suppression of the expression of the target gene to a very strong or even a 100% suppression of the expression of the target gene. However, a person skilled in the art will be able to select those plant cells, plants, events or plant lines leading to the desired degree of silencing and desired phenotype.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region, which is functionally or structurally defined, may comprise additional DNA regions etc.

The following non-limiting Examples describe chimeric genes for the alteration of the level of beta-1,2-xylose residues on protein-bound N-glycans in Nicotiana species, particularly in Nicotiana benthamiana, and in Nicotiana cultivars, particularly in Nicotiana tabacum cv. Petite Havana SR1, and uses thereof. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

Throughout the description and Examples, reference is made to the following sequences represented in the sequence listing:
SEQ ID NO: 1: nucleotide sequence of the oligonucleotide XylF4 suitable to amplify a part of a Nicotiana XylT gene or cDNA.
SEQ ID NO: 2: nucleotide sequence of the oligonucleotide XylR4 suitable to amplify a part of a Nicotiana XylT gene or cDNA.
SEQ ID NO: 3: partial cDNA sequence of Nicotiana tabacum cv. Petite Havana SR1 XylT gene variant 1.
SEQ ID NO: 4: partial amino acid sequence of Nicotiana tabacum cv. Petite Havana SR1 XylT protein variant 1.
SEQ ID NO: 5: partial cDNA sequence of Nicotiana tabacum cv. Petite Havana SR1 XylT gene variant 2.
SEQ ID NO: 6: partial amino acid sequence of Nicotiana tabacum cv. Petite Havana SR1 XylT protein variant 2.
SEQ ID NO: 7: partial nucleotide sequence of Nicotiana tabacum cv. Petite Havana SR1 XylT gene variant 1.
SEQ ID NO: 8: partial amino acid sequence of Nicotiana tabacum cv. Petite Havana SR1 XylT protein variant 1.
SEQ ID NO: 9: partial nucleotide sequence of Nicotiana tabacum cv. Petite Havana SR1 XylT gene variant 2.
SEQ ID NO: 10: partial amino acid sequence of Nicotiana tabacum cv. Petite Havana SR1 XylT protein variant 2.
SEQ ID NO: 11: partial nucleotide sequence of Nicotiana benthamiana XylT gene variant 1.
SEQ ID NO: 12: partial amino acid sequence of Nicotiana benthamiana XylT protein variant 1.
SEQ ID NO: 13: partial nucleotide sequence of Nicotiana benthamiana XylT gene variant 2.
SEQ ID NO: 14: partial amino acid sequence of Nicotiana benthamiana XylT protein variant 2.
SEQ ID NO: 15: nucleotide sequence of the oligonucleotide XylF8 suitable to amplify a part of a Nicotiana tabacum cv. Petite Havana SR1 XylT gene or cDNA.
SEQ ID NO: 16: nucleotide sequence of the oligonucleotide XylR8 suitable to amplify a part of a Nicotiana tabacum cv. Petite Havana SR1 XylT gene or cDNA.
SEQ ID NO: 17: partial cDNA sequence of Nicotiana tabacum cv. Petite Havana SR1 XylT gene variant 1.
SEQ ID NO: 18: nucleotide sequence of T-DNA region of vector pTKW20.
SEQ ID NO: 19: nucleotide sequence of the oligonucleotide XylF9 suitable to amplify a part of a Nicotiana benthamiana XylT gene or cDNA.
SEQ ID NO: 20: nucleotide sequence of the oligonucleotide XylR9 suitable to amplify a part of a Nicotiana benthamiana XylT gene or cDNA.
SEQ ID NO: 21: partial sequence of Nicotiana benthamiana XylT gene variant 1.
SEQ ID NO: 22: nucleotide sequence of T-DNA region of vector pTKW29.
SEQ ID NO: 23: Nicotiana tabacum cv. Xanthi mRNA for putative beta-(1,2)-xylosyltransferase (accession number AJ627182)
SEQ ID NO: 24: putative beta-(1,2)-xylosyltransferase encoded by SEQ ID NO:23
SEQ ID NO: 25: Nicotiana tabacum cv. Xanthi xylt gene for putative beta-(1,2)-xylosyltransferase (accession number AJ627183)
SEQ ID NO: 26: putative beta-(1,2)-xylosyltransferase encoded by SEQ ID NO:25

### EXAMPLES

### Example 1

### Design of Degenerated Primers for the Isolation of XylT cDNA and Gene Sequences from Nicotiana tabacum cv. Petite Havana SR1 and Nicotiana benthamiana

Oligonucleotide sequences to be used as degenerated primers in a PCR amplification of XylT cDNA and genomic DNA from Nicotiana tabacum cv. Petite Havana SR1 and Nicotiana benthamiana were designed based on exon sequences of a genomic DNA sequence from Nicotiana tabacum cv. Xanthi encoding a putative XylT protein (accession number AJ627183). The forward primer (SEQ ID NO:1) was designed with CACC at its 5′ end for cloning purposes. In this way the following degenerated primers were generated:

(SEQ ID NO: 1)

| | |
|---|---|
| XylF4: | 5′-CACCTTGTTTCTCTCTTCGCTCTCAACTCAATCACTC-3′ |

(SEQ ID NO: 2)

| | |
|---|---|
| XylR4: | 5′-TCGATCACAACTGGAGGATCCGCATAA-3′ |

### Example 2

### Isolation of XylT cDNA Sequences from Nicotiana tabacum cv. Petite Havana SR1

The degenerated primers described in Example 1 were used to isolate XylT cDNA sequences from Nicotiana tabacum cv. Petite Havana SR1:

RNA was extracted from leaves of Nicotiana tabacum cv. Petite Havana SR1 using the RNeasy Plant Mini Kit (Qiagen) according to the manufacturer's protocol and used for cDNA synthesis using SuperScript™ First-strand synthesis System for RT-PCR (Invitrogen Life Technologies) according to the manufacturer's instructions.

Using the cDNA as template and primer pair XylF4/XylR4, PCR amplification was performed under the following conditions: 15 sec at 94° C. (denaturation) and 3 min at 68° C. for 40 cycles (annealing and elongation).

A DNA fragment of about 1500 basepairs was amplified, cloned into a pENTR™/D-TOPO® vector (Invitrogen) and several clones were sequenced (comprising the sequences of SEQ ID NO: 3—XylTc2Nt—and SEQ ID NO: 5—XylTc7Nt).

An alignment between a mRNA sequence from Nicotiana tabacum cv. Xanthi encoding a putative XylT protein (accession number AJ627182; SEQ ID NO:23) and the XylT cDNA sequences isolated from Nicotiana tabacum cv. Petite Havana SR1 (SEQ ID NO: 3 and 5) is shown in FIG. 1.

An alignment between the putative XylT protein encoded by the mRNA sequence from Nicotiana tabacum cv. Xanthi (accession number AJ627182; SEQ ID NO:24) and by the cDNA sequences isolated from Nicotiana tabacum cv. Petite Havana SR1 (SEQ ID NO: 4 and 6) is shown in FIG. 2.

### Example 3

### Isolation of XylT Gene Sequences of Nicotiana tabacum cv. Petite Havana SR1 and of Nicotiana benthamiana

The degenerated primers described in Example 1 were used to isolate XylT gene sequences from Nicotiana tabacum cv. Petite Havana SR1 and from Nicotiana benthamiana:

DNA was extracted from leaves of Nicotiana tabacum cv. Petite Havana SR1 and of Nicotiana benthamiana based on the protocol described by Bernatzky and Tanksley (1986).

Using the genomic DNA as template and primer pair XylF4/XylR4, PCR amplification was performed under the following conditions: 15 sec at 94° C. (denaturation) and 4 min 30 sec at 68° C. for 40 cycles (annealing and elongation).

Using the genomic DNA from Nicotiana tabacum cv. Petite Havana SR1 as template for the PCR amplification, a DNA fragment of about 3400 basepairs was amplified, cloned into a pENTR™/D-TOPO® vector (Invitrogen) and several clones were sequenced (comprising the sequences of SEQ ID NO: 7—XylTg1Nt—and SEQ ID NO: 9—XylTg3Nt).

The XylT genomic DNA sequences XylTg1Nt and XylTg3Nt comprise two putative intron sequences and three putative exon sequences. The location of the intron sequences are:
For XylTg1Nt: from the nucleotide at position 679 to the nucleotide at position 1974 and from the nucleotide at position 2125 to the nucleotide at position 2722 of SEQ ID NO: 7, and
For XylTg3Nt: from the nucleotide at position 658 to the nucleotide at position 1953 and from the nucleotide at position 2104 to the nucleotide at position 2701 of SEQ ID NO: 9.

Using the genomic DNA from Nicotiana benthamiana as template for the PCR amplification, a DNA fragment of between about 3300 and about 3600 basepairs was amplified, cloned into a pENTR™/D-TOPO® vector (Invitrogen) and several clones were sequenced (comprising the sequences of SEQ ID NO: 11—XylTg14Nb—and SEQ ID NO:13—XylTg19Nb).

The XylT genomic DNA sequences XylTg14Nb and XylTg19Nb comprise two putative intron sequences and three putative exon sequences. The location of the intron sequences is:
XylTg14Nb from the nucleotide at position 658 to the nucleotide at position 1917 and from the nucleotide at position 2068 to the nucleotide at position 2612 of SEQ ID NO: 11, and
XylTg19Nb from the nucleotide at position 649 to the nucleotide at position 2194 and from the nucleotide at position 2345 to the nucleotide at position 2888 of SEQ ID NO: 13.

An alignment between the genomic DNA sequence from Nicotiana tabacum cv. Xanthi encoding a putative XylT protein (accession number AJ627183; SEQ ID NO:25) and the XylT genomic DNA sequences isolated from Nicotiana tabacum cv. Petite Havana SR1 (SEQ ID NO: 7 and 9) and from Nicotiana benthamiana (SEQ ID NO: 11 and 13) is shown in FIG. 3.

An alignment between the putative XylT protein encoded by the genomic DNA sequence from Nicotiana tabacum cv. Xanthi (accession number AJ627183; SEQ ID NO:26) and by the genomic DNA sequences isolated from Nicotiana tabacum cv. Petite Havana SR1 (SEQ ID NO:8 and 10) and from Nicotiana benthamiana (SEQ ID NO: 12 and 14) is shown in FIG. 4.

### Example 4

### Construction of a T-DNA Vector Containing a Nicotiana XylT Silencing Gene

DNA fragments amplified from Nicotiana XylT sequences described in Examples 2 and 3 were used to construct T-DNA vectors comprising a chimeric gene which upon transcription yields an RNA molecule comprising a sense and antisense DNA sequence from the amplified DNA fragment, and which could basepair to form a double stranded RNA molecule. Such chimeric genes can be used to reduce the expression of a XylT gene in Nicotiana, particularly in Nicotiana tabacum cv. Petite Havana SR1 and Nicotiana benthamiana.

4.1, Construction of a T-DNA vector comprising a XylT silencing gene with a DNA fragment amplified from a XylT sequence from Nicotiana tabacum cv. Petite Havana SR1 Oligonucleotide sequences to be used as non-degenerated primers in a PCR amplification of a XylT cDNA sequence from Nicotiana tabacum cv. Petite Havana SR1 were designed based on the cDNA sequence from Nicotiana tabacum cv. Petite Havana SR1 isolated in Example 2. The forward primer (SEQ ID NO:15) was designed with CACC at its 5′ end for cloning purposes. In this way the following non-degenerated primers were generated:

| (SEQ ID NO: 15) | |
|---|---|
| XylF8: | 5′-CACCTCTCGCCTTTGGGATATGAAACT-3′ |
| (SEQ ID NO: 16) | |
| XylR8: | 5′-ACAGCTTTGGTGCTGCAGAAACT-3′ |

Using the vector comprising a DNA fragment amplified from a XylT cDNA sequence of Nicotiana tabacum cv. Petite Havana SR1 as described in Example 2 (SEQ ID NO:3-XylTc2Nt) as template and primer pair XylF8/XylR8, a PCR amplification was performed under the following conditions: 15 sec at 94° C. (denaturation), 30 sec at 56° C. (annealing) and 45 sec at 68° C. (elongation) for 25 cycles.

A DNA fragment of about 470 bp (XylTi4Nt; SEQ ID NO: 17) was amplified and cloned into a pENTR™/D-TOPO® vector (Invitrogen) yielding plasmid pKW19. Plasmid pKW19 was recombined with pHellsgate12 (Helliwell and Waterhouse, Methods (2003) 30: 289-295) using Gateway® LR Clonase™ II (Invitrogen) yielding plasmid pTKW20.

The T-DNA sequence of pTKW20 (SEQ ID NO: 18) thus comprises:
A chimeric XylT silencing gene comprising:
   a fragment including the promoter region of the Cauliflower Mosaic Virus 35S transcript (Odell et al., 1985)
   (SEQ ID NO:18 from nucleotide 969 to nucleotide 2314)
   a fragment including a part of the Nicotiana tabacum cv. Petite Havana SR1XylT cDNA sequence cloned in sense orientation
   (SEQ ID NO:18 from nucleotide 2365 to nucleotide 2834)
   a fragment containing the intron of the catase-1 gene from castor bean
   (SEQ ID NO:18 from nucleotide 2893 to nucleotide 3088)
   a fragment containing the second intron of the pyruvate orthophosphate dikinase gene from Flaveria trinervia as described by Rosche and Westhoff (1995) in reverse orientation
   (SEQ ID NO:18 from nucleotide 3130 to nucleotide 3871).
   a fragment including a part of the Nicotiana tabacum cv. Petite Havana SR1 XylT cDNA sequence cloned in antisense orientation
   (SEQ ID NO:18 from nucleotide 3957 to nucleotide 4426).
   a fragment including the 3′ untranslated region of the octopine synthase gene of Agrobacterium tumefaciens as described by De Greve et al. (1982)
   (SEQ ID NO:18 from nucleotide 4479 to nucleotide 5244).
A chimeric gene encoding a selectable marker comprising:
   a fragment including the promoter region of the nopaline synthase gene of Agrobacterium tumefaciens T-DNA
   (SEQ ID NO:18 from nucleotide 5512 to nucleotide 5744).
   a fragment including the nptII antibiotic resistance gene
   (SEQ ID NO:18 from nucleotide 5745 to nucleotide 6690).
   A fragment including the 3′ untranslated region of the nopaline synthase gene of A. tumefaciens T-DNA.
   (SEQ ID NO:18 from nucleotide 6691 to nucleotide 7396).

The T-DNA vector was introduced into Agrobacterium tumefaciens comprising a helper Ti-plasmid.

4.2. Construction of a T-DNA vector comprising a XylT silencing gene with a DNA fragment amplified from a XylT sequence from Nicotiana benthamiana

Oligonucleotide sequences to be used as non-degenerated primers in a PCR amplification of a XylT gene sequence from Nicotiana benthamiana were designed based on the gene sequence from Nicotiana benthamiana isolated in Example 3. The forward primer (SEQ ID NO:19) was designed with GGCCGGATCCTCG at its 5′ end and the reverse primer (SEQ ID NO:20) was designed with GGCCATCGATGGTACC at its 5′ end for cloning purposes. In this way the following non-degenerated primers were generated:

XylF9:
(SEQ ID NO: 19)
5′-GGCCGGATCCTCGAGACACAATTGGAGGAAACATGGAAAGC-3′
XylR9:
(SEQ ID NO: 20)
5′-GGCCATCGATGGTACCGGCCCAGCTCTTTATGGAATCAAA-3′

Using the vector comprising a DNA fragment amplified from a XylT gene sequence of Nicotiana benthamiana as described in Example 3 (SEQ ID NO:11—XylTg14Nb) as template and primer pair XylF9/XylR9, a PCR amplification was performed under the following conditions: 15 sec at 94° C. (denaturation), 30 sec at 58° C. (annealing) and 30 sec at 68° C. (elongation) for 25 cycles.

A DNA fragment of about 430 bp (XylTiNb; SEQ ID NO: 21) was amplified and digested with XhoI and KpnI and with BamHI and ClaI, respectively. The XhoI/KpnI and the BamHI/ClaI digested fragments were cloned in pHANNIBAL (Helliwell and Waterhouse, 2003) digested with XhoI/KpnI and BamHI/ClaI yielding pKW28.

Plasmid pKW28 thus comprises a chimeric XylT silencing gene comprising:
a fragment including the promoter region of the Cauliflower Mosaic Virus 35S transcript (Odell et al., 1985)
(SEQ ID NO:22 from nucleotide 3779 to nucleotide 2434)
a fragment including a part of the Nicotiana benthamiana XylT gene sequence cloned in sense orientation
(SEQ ID NO:22 from nucleotide 2427 to nucleotide 2023).
a fragment containing the second intron of the pyruvate orthophosphate dikinase gene from Flaveria trinervia as described by Rosche and Westhoff (1995)
(SEQ ID NO:22 from nucleotide 1991 to nucleotide 1250).
a fragment including a part of the Nicotiana benthamiana XylT gene sequence cloned in antisense orientation
(SEQ ID NO:22 from nucleotide 1211 to nucleotide 807).
a fragment including the 3′ untranslated region of the octopine synthase gene of Agrobacterium tumefaciens as described by De Greve et al. (1982)
(SEQ ID NO:22 from nucleotide 786 to nucleotide 76).
   between restriction sites MscI and PstI.

Plasmid pKW28 is digested with MscI and PstI and the chimeric gene is introduced between the T-DNA borders of a T-DNA vector cut with PstI and SmaI together with a chimeric gene encoding a selectable marker comprising:
a fragment including the promoter region of the nopaline synthase gene of A. tumefaciens T-DNA
(SEQ ID NO:22 from nucleotide 3854 to nucleotide 4140).
a fragment including the bar phosphinothricin resistance gene (De Block et al., 1987)
(SEQ ID NO:22 from nucleotide 4161 to nucleotide 4712).
a fragment including the 3′ untranslated region of the nopaline synthase gene of A. tumefaciens T-DNA
(SEQ ID NO:22 from nucleotide 4731 to nucleotide 4991).
   to yield pTKW29 (sequence of the T-DNA of pTKW29 is represented in SEQ ID NO: 22).

The vector pTKW29 is derived from pGSC1700 (Cornelissen and Vandewiele, 1989). The vector backbone contains the following genetic elements:
the plasmid core comprising the origin of replication from the plasmid pBR322 (Bolivar et al., 1977) for replication in Escherichia coli (ORI ColE1) and a restriction fragment comprising the origin of replication from the Pseudomonas plasmid pVS1 (Itoh et al., 1984) for replication in Agrobacterium tumefaciens (ORI pVS1).
a selectable marker gene conferring resistance to streptomycin and spectinomycin (aadA) for propagation and selection of the plasmid in Escherichia coli and Agrobacterium tumefaciens.
a DNA region consisting of a fragment of the neomycin phosphotransferase coding sequence of the nptI gene from transposon Tn903 (Oka et al., 1981).

The T-DNA vector is introduced into Agrobacterium tumefaciens comprising a helper Ti-plasmid.

### Example 5

### Analysis of Transgenic Nicotiana Plants Harboring a XylT Silencing Gene

Nicotiana plants were transformed using the Agrobacterium tumefaciens strains described in Example 4:

5.1. Analysis of transgenic Nicotiana tabacum cv. Petite Havana SR1 plants harboring a XylT silencing gene

Nicotiana tabacum cv. Petite Havana SR1 plants were transformed using the Agrobacterium tumefaciens strain described in Example 4.1. according to the protocol as described in Zambryski et al. (1983). Fifty-two transgenic Nicotiana tabacum lines, comprising the chimeric genes as described in Example 4.1. were obtained.

Transgenic plant lines were analyzed on molecular level using Southern blot analysis. Similarly, the plant lines are analyzed for XylT RNA expression using Northern blot analysis.

An indication of XylT activity can be obtained by comparing the level of beta-1,2-xylose residues present on the glycans of proteins from the transgenic lines with that of untransformed plants. The level of beta-1,2-xylose residues on protein-bound N-glycans of plants can be measured e.g. by Western blot analysis using xylose-specific antibodies as described e.g. by Faye et al. (1993) or by mass spectrometry on glycans isolated from the plant's glycoproteins using Matrix-assisted Laser Desorption/Ionization Time-of-Flight mass spectronomy (MALDI-TOF-MS) as described e.g. by Kolarich and Altmann (2000) or using Liquid Chromatography Tandem mass spectronomy (LC/MS/MS) as described e.g. by Henriksson et al. (2003).

5.2. Analysis of transgenic Nicotiana benthamiana plants harboring a XylT silencing gene

Similarly, Nicotiana benthamiana plants were transformed using the Agrobacterium tumefaciens strain described in Example 4.2. and the expression of XylT and the level of beta-1,2-xylose residues present on the glycans of proteins was analyzed as described above.

Fifty four transgenic Nicotiana benthamiana lines comprising the chimeric genes described in Example 4.2. were obtained after leaf disk transformation with pTKW29.

To determine the level of beta-1,2-xylose residues present on the glycans of endogenous proteins of these plant lines, soluble leaf proteins of each individual were analyzed by Western blot using a beta-1,2-xylose-specific antibody.

Six samples showed very weak reaction with the antibody and six samples had no detectable reaction with the antibody. For the other samples, the level of reaction with the antibody ranged from weak to wild-type level.

To determine the number of insertions of the chimeric XylT silencing gene from pTKW29, genome DNA from the plant lines showing very weak or negative reactions to the beta-1,2-xylose-specific antibody was isolated, digested with EcoRI and analyzed by Southern blot using a probe spanning the 35S promoter region and a probe spanning the bar phosphinotricin resistance gene's coding region.

None of the twelve plant lines showed a single insertion. One plant line contained two insertions and was negative for xylose using Western blot analysis.

To test whether these two chimeric XylT silencing genes inserted independently and to obtain plants which are negative for xylose on Western blot and which contain a single chimeric XylT silencing gene, progeny resulting from self fertilization of the plant line containing two insertions were sown and twenty five plant lines were analyzed by Western blot analysis.

### REFERENCES

Baulcombe (2004). Nature 431: 356-363.
Bernatzky and Tanksley (1986). Theor. Appl. Genet. 72: 314-321.
Bolivar et al. (1977). Gene 2: 95-113.
Cornelissen and Vandewiele (1989). Nucleic Acids Research 17: 19-25.
De Block et al. (1984). EMBO J. 3(8):1681-1689
De Block et al. (1987). EMBO J 6:2513.
De Greve et al. (1982). J. Mol. Appl. Genetics 1 (6): 499-511.
Faye et al. (1993). Analytical Biochemistry 209: 104-108.
Fischer et al. (2004). Curr. Opin. Plant Biol. 7:152-158.
Helliwell and Waterhouse (2003). Methods 30: 289-295.
Henikoff and Henikoff (1992). Proc Natl Acad Sci USA 89(22):10915-10919.
Henriksson et al. (2003). Biochem. J. 375: 61-73.
Horsch et al. (1985). Science 227: 1229-1231.
Itoh et al. (1984). Plasmid 11: 206.
Khoo et al. (1997). Glycobiology 7: 663-677.
Kolarich and Altmann (2000). Anal. Biochem. 285: 64-75.
Mulder et al. (1995). Eur. J. Biochem. 232: 272-283.
Odell et al. (1985). Nature 313: 810.
Oka et al. (1981). Journal of Molecular Biology, 147, 217-226.
Rosche and Westhoff (1995). Plant Molecular Biology 29 (4): 663-678.
Stoger et al. (2002). Curr. Opin. Biotechnol. 13: 161-166.
Strasser et al. (2000). FEBS Lett. 472:105-108.
Strasser et al. (2004a). 2^{nd} EPSO Conference, October 2004, Italy, Abstract book p. 56, S036.
Strasser et al. (2004b). FEBS Lett. 561:132-136.
Tezuka et al. (1992). Eur J. Biochem. 203(3):401-413.
Twyman et al. (2003). Trends Biotechnol. 21: 570-578.
Zambryski et al. (1983). EMBO J. 2: 2143-2150).
Zeng et al. (1997). J. Biol. Chem. 272: 31340-31347.

1. A method to produce a Nicotiana plant cell or plant having a low level of beta-1,2-xylose residues on protein-bound N-glycans comprising the steps of
a) introducing a chimeric gene into plant cells of a Nicotiana species or cultivar to generate transgenic plant cells, said chimeric gene comprising the following operably linked DNA fragments:
i) a plant expressible promoter;
ii) a transcribable DNA region comprising
(1) a first sense DNA region comprising a nucleotide sequence of at least 19 out of 20 consecutive nucleotides from a nucleotide sequence encoding a Nicotiana XylT protein or the complement thereof, wherein said at least 19 out of 20 consecutive nucleotides encode at least one Nicotiana species- or cultivar-specific XylT amino acid, or from a nucleotide sequence of a Nicotiana XylT gene or a Nicotiana XylT cDNA or the complement thereof wherein said at least 19 out of 20 consecutive nucleotides comprise at least one Nicotiana species-specific XylT nucleotide;
(2) a second antisense DNA region comprising a nucleotide sequence of at least 19 consecutive nucleotides which have at least 95% sequence identity to the complement of said first DNA region;
wherein an RNA molecule transcribed from said transcribable DNA region is capable of forming a double stranded RNA region at least between an RNA region transcribed from said first sense DNA region and an RNA region transcribed from said second antisense DNA region; and
iii) a DNA region comprising a transcription termination and polyadenylation signal functional in plants;
b) optionally, identifying a transgenic Nicotiana plant cell which has a lower level of beta-1,2-xylose residues on protein-bound N-glycans than an untransformed Nicotiana plant cell;
c) optionally, regenerating said transgenic Nicotiana plant cells to obtain transgenic Nicotiana plants; and
d) optionally, identifying a transgenic Nicotiana plant which has a lower level of beta-1,2-xylose residues on protein-bound N-glycans than an untransformed Nicotiana plant.

2. The method of claim 1, wherein said Nicotiana species-specific XylT amino acid is a Nicotiana benthamiana-specific XylT amino acid.

3. The method of claim 1, wherein said nucleotide sequence encoding a Nicotiana XylT protein comprises a nucleotide sequence encoding the amino acid sequence of SEQ ID No.: 12 or SEQ ID No.:14.

4. The method of claim 1, wherein said Nicotiana species-specific XylT nucleotide is a Nicotiana benthamiana-specific XylT nucleotide.

5. The method of claim 1, wherein said nucleotide sequence of said Nicotiana XylT gene comprises the nucleotide sequence of SEQ ID No.: 11, SEQ ID No.:13, or SEQ ID No. 21.

6. The method of claim 1, wherein said Nicotiana cultivar-specific XylT amino acid is a Nicotiana tabacum cv. Petite Havana SR1-specific XylT amino acid.

7. The method of claim 1, wherein said nucleotide sequence encoding said Nicotiana XylT protein comprises a nucleotide sequence encoding the amino acid sequence of SEQ ID No.: 4, SEQ ID No.:6, SEQ ID No.: 8 or SEQ ID No.:10.

8. The method of claim 1, wherein said Nicotiana cultivar-specific XylT nucleotide is a Nicotiana tabacum cv. Petite Havana SR1-specific XylT nucleotide.

9. The method of claim 1, wherein said nucleotide sequence of said Nicotiana XylT gene or said Nicotiana XylT cDNA comprises the nucleotide sequence of SEQ ID No.: 3, SEQ ID No.: 5, SEQ ID No.: 8, SEQ ID No.:10, or SEQ ID No.: 17.

10. The method of claim 1, wherein said first and said second DNA region comprise at least 50 consecutive nucleotides.

11. The method of claim 1, wherein said first and said second DNA region comprise at least 200 consecutive nucleotides.

12. A method to produce a Nicotiana plant cell or plant having a low level of beta-1,2-xylose residues on protein-bound N-glycans comprising the steps of:
a) providing one or more double stranded RNA molecules to plant cells or plants of a Nicotiana species or cultivar, wherein the double stranded RNA molecules comprise two RNA strands, one RNA strand consisting essentially of an RNA nucleotide sequence of 19 out of 20 to 21 consecutive nucleotides from a nucleotide sequence encoding a Nicotiana XylT protein, or the complement thereof, wherein said 19 out of 20 to 21 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 to 21 consecutive nucleotides comprise at least one Nicotiana species- or cultivar-specific XylT nucleotide;
b) identifying a Nicotiana plant cell or plant comprising said double stranded RNA molecule or molecules which has a lower level of beta-1,2-xylose residues on protein-bound N-glycans than a same Nicotiana plant cell or plant which does not comprise said double stranded RNA molecule or molecules.

13. The method of claim 12, wherein said double stranded RNA is provided to said plant cells or plants by integrating a chimeric gene into the genome of plant cells of said Nicotiana species or cultivar to generate transgenic plant cells and, optionally, regenerating said plant cells to obtain transgenic plants, said chimeric gene comprising the following operably linked DNA fragments:
a) a plant expressible promoter;
b) a DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in antisense orientation;
c) a DNA region comprising a transcription termination and polyadenylation signal functional in plants.

14. The method of claim 12, wherein said double stranded RNA is provided to said plant cells or plants by integrating a chimeric gene into the genome of said plant cells to generate transgenic plant cells and, optionally, regenerating said plant cells to obtain transgenic plants, said chimeric gene comprising the following operably linked DNA fragments:
a) a plant expressible promoter;
b) a DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid it complements, or selected from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in sense orientation;
c) a DNA region comprising a transcription termination and polyadenylation signal functional in plants.

15. The method of claim 12, wherein said double stranded RNA is provided to said plant cells or plants by integrating a chimeric gene into the genome of said plant cells to generate transgenic plant cells and, optionally, regenerating said plant cells to obtain transgenic plants, said chimeric gene comprising the following operably linked DNA fragments:
a) a plant expressible promoter;
b) a transcribable DNA region comprising:
i) a first DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in antisense orientation;
ii) a second DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in sense orientation,
whereby an RNA molecule produced by transcription of said transcribable DNA region is capable of forming a double stranded RNA region by base-pairing at least between an RNA region corresponding to said first DNA region and an RNA region corresponding to said second RNA region; and
c) a DNA region comprising a transcription termination and polyadenylation signal functional in plants.

16. The method of claim 12, wherein said Nicotiana species-specific XylT amino acid is a Nicotiana benthamiana-specific XylT amino acid.

17. The method of claim 12, wherein said nucleotide sequence encoding a Nicotiana XylT protein comprises a nucleotide sequence encoding the amino acid sequence of SEQ ID No.: 12 or SEQ ID No.:14.

18. The method of claim 12, wherein said Nicotiana species-specific XylT nucleotide is a Nicotiana benthamiana-specific XylT nucleotide.

19. The method of claim 12, wherein said nucleotide sequence of said Nicotiana XylT gene comprises the nucleotide sequence of SEQ ID No.: 11, SEQ ID No.:13, or SEQ ID No. 21.

20. The method of claim 12, wherein said Nicotiana cultivar-specific XylT amino acid is a Nicotiana tabacum cv. Petite Havana SR1-specific XylT amino acid.

21. The method of claim 12, wherein said nucleotide sequence encoding said Nicotiana XylT protein comprises a nucleotide sequence encoding the amino acid sequence of SEQ ID No.: 4, SEQ ID No.:6, SEQ ID No.: 8 or SEQ ID No.:10.

22. The method of claim 12, wherein said Nicotiana cultivar-specific XylT nucleotide is a Nicotiana tabacum cv. Petite Havana SR1-specific XylT nucleotide.

23. The method of claim 12, wherein said nucleotide sequence of said Nicotiana XylT gene or said Nicotiana XylT cDNA comprises the nucleotide sequence of SEQ ID No.: 3, SEQ ID No.: 5, SEQ ID No.: 8, SEQ ID No.:10, or SEQ ID No.: 17.

24. A method to produce a Nicotiana plant cell or plant having a low level of beta-1,2-xylose residues on protein-bound N-glycans comprising the steps of:
a) identifying a fragment of a XylT protein encoding DNA sequence obtainable from a first Nicotiana species or cultivar, comprising using
i) a probe comprising a DNA fragment comprising a nucleotide sequence encoding the amino acid sequence of SEQ ID No.: 4, SEQ ID No.:6, SEQ ID No.: 8, SEQ ID No.:10, SEQ ID No.: 12, or SEQ ID No.:14;
ii) a probe comprising a DNA fragment comprising the nucleotide sequence of any one of SEQ ID No.:-3, SEQ ID No.: 5, SEQ ID No.: 7, SEQ ID No.: 9, SEQ ID No.: 11, SEQ ID No.: 13, SEQ ID No.: 17, or SEQ ID No.: 21;
iii) a probe comprising a DNA fragment or oligonucleotide comprising a nucleotide sequence consisting of between 20 to 1513 consecutive nucleotides from a nucleotide sequence encoding the amino acid sequence of SEQ ID No.: 4, or SEQ ID No.:6;
iv) a probe comprising a DNA fragment or oligonucleotide comprising a nucleotide sequence consisting of between 20 to 3574 consecutive nucleotides from a nucleotide sequence encoding the amino acid sequence of SEQ ID No.: 8, SEQ ID No.:10, SEQ ID No.: 12, or SEQ ID No.:14;
v) a probe comprising a DNA fragment or oligonucleotide comprising a nucleotide sequence consisting of between 20 to 3574 consecutive nucleotides from a nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21;
vi) a PCR primer comprising an oligonucleotide sequence comprising a nucleotide sequence comprising between 20 to 200 consecutive nucleotides from a nucleotide sequence encoding the amino acid sequence of SEQ ID No.: 4, or SEQ ID No.:6;
vii) a PCR primer comprising an oligonucleotide sequence comprising a nucleotide sequence comprising between 20 to 200 consecutive nucleotides from a nucleotide sequence encoding the amino acid sequence of SEQ ID No.: 8, SEQ ID No.:10, SEQ ID No.: 12, or SEQ ID No.:14;
viii) a PCR primer comprising an oligonucleotide sequence comprising a nucleotide sequence comprising between 20 to 200 consecutive nucleotides from the nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21; or
ix) a PCR primer comprising an oligonucleotide comprising the nucleotide sequence of any one of SEQ ID No.: 1, SEQ ID No.: 2, SEQ ID No.: 15 or SEQ ID No.: 16, SEQ ID No.:19 or SEQ ID No.20;
b) providing one or more double stranded RNA molecules to plant cells or plants of said first or a second Nicotiana species or cultivar, wherein said double stranded RNA molecules comprise two RNA strands, one RNA strand consisting essentially of an RNA nucleotide sequence of 20 to 21 consecutive nucleotides from a nucleotide sequence of said XylT protein encoding DNA fragment, or the complement thereof, wherein said 20 to 21 consecutive nucleotides encode at least one Nicotiana species- or cultivar-specific XylT amino acid, respectively, or wherein said 20 to 21 consecutive nucleotides comprise at least one Nicotiana species- or cultivar-specific XylT nucleotide, respectively; and
c) identifying a Nicotiana plant cell or plant comprising said double stranded RNA molecule or molecules which has a lower level of beta-1,2-xylose residues on protein-bound N-glycans than a same Nicotiana plant cell or plant, which does not comprise said double stranded RNA molecule or molecules.

25. The method of claim 24, wherein provision of said double stranded RNA molecule or molecules is achieved by providing to said plant cells or plants a double stranded RNA molecule or molecules comprising a first nucleotide sequence of at least 19 out of 20 consecutive nucleotides from the nucleotide sequence of said XylT protein encoding DNA fragment, or the complement thereof, wherein said at least 19 out of 20 consecutive nucleotides encode at least one Nicotiana species- or cultivar-specific XylT amino acid, or wherein said at least 19 out of 20 consecutive nucleotides comprise at least one Nicotiana species- or cultivar-specific XylT nucleotide, and a second nucleotide sequence which is the complement of said first nucleotide sequence.

26. The method of claim 24, wherein said double stranded RNA molecules are provided to said plant cells or plants by integrating a chimeric DNA into the genome of said plant cells to generate transgenic plant cells and, optionally, regenerating said plant cells to obtain transgenic plants, said chimeric DNA comprising the following operably linked DNA fragments:
a) a plant expressible promoter;
b) a transcribable DNA region comprising
i) a first sense DNA region comprising a nucleotide sequence of at least 19 out of 20 consecutive nucleotides from the nucleotide sequence of said XylT protein encoding DNA fragment, or the complement thereof, wherein said at least 19 out of 20 consecutive nucleotides encode at least one Nicotiana species- or cultivar-specific XylT amino acid, respectively, or wherein said at least 19 out of 20 consecutive nucleotides comprise at least one Nicotiana species- or cultivar-specific XylT nucleotide, respectively;
ii) a second antisense DNA region comprising a nucleotide sequence of at least 19 consecutive nucleotides which have at least 95% sequence identity to the complement of said first DNA region;
wherein an RNA molecule transcribed from said transcribable region is capable of forming a double stranded RNA region at least between an RNA region transcribed from said first sense DNA region and an RNA region transcribed from said second antisense DNA region; and
c) a DNA region comprising a transcription termination and polyadenylation signal functional in plants.

27. The method of claim 1 further comprising the step of crossing said Nicotiana plant having a low level of beta-1,2-xylose residues on protein-bound N-glycans to another Nicotiana plant to obtain Nicotiana progeny plants having a low level of beta-1,2-xylose residues on protein-bound N-glycans.

28. A method to identify a Nicotiana XylT DNA fragment, comprising the steps of
a) providing genomic DNA or cDNA obtainable from a Nicotiana species or cultivar;
b) using:
i) a probe comprising a DNA fragment comprising a nucleotide sequence encoding the amino acid sequence of SEQ ID No.: 4, SEQ ID No.:6, SEQ ID No.: 8, SEQ ID No.:10, SEQ ID No.: 12, or SEQ ID No.:14;
ii) a probe comprising a DNA fragment comprising the nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21;
iii) a probe comprising a DNA fragment or oligonucleotide comprising a nucleotide sequence consisting of between 20 to 1513 consecutive nucleotides from a nucleotide sequence encoding the amino acid sequence of SEQ ID No.: 4, or SEQ ID No.:6;
iv) a probe comprising a DNA fragment or oligonucleotide comprising a nucleotide sequence consisting of between 20 to 3574 consecutive nucleotides from a nucleotide sequence encoding the amino acid sequence of SEQ ID No.: 8, SEQ ID No.:10, SEQ ID No.: 12, or SEQ ID No.:14
v) a probe comprising a DNA fragment or oligonucleotide comprising a nucleotide sequence consisting of between 20 to 3574 consecutive nucleotides from a nucleotide sequence of any one of SEQ ID No.: 3, SEQ ID No.: 5, SEQ ID No.: 7, SEQ ID No.: 9, SEQ ID No.: 11, SEQ ID No.: 13, SEQ ED No.: 17, or SEQ ID No.: 21;
vi) a PCR primer comprising an oligonucleotide sequence comprising a nucleotide sequence comprising between 20 to 200 consecutive nucleotides from a nucleotide sequence encoding the amino acid sequence of SEQ ID No.: 4, or SEQ ID No.:6;
vii) a PCR primer comprising an oligonucleotide sequence comprising a nucleotide sequence comprising between 20 to 200 consecutive nucleotides from a nucleotide sequence encoding the amino acid sequence of SEQ ID No.: 8, SEQ ID No.:10, SEQ ID No.: 12, or SEQ ID No.:14;
viii) a PCR primer comprising an oligonucleotide sequence comprising a nucleotide sequence comprising between 20 to 200 consecutive nucleotides from the nucleotide sequence of any one of SEQ ID No.: 3, SEQ ID No.: 5, SEQ ID No.: 7, SEQ ID No.: 9, SEQ ID No.: 11, SEQ ID No.: 13, SEQ ID No.: 17, or SEQ ID No.: 21; or
ix) a PCR primer comprising an oligonucleotide sequence comprising the nucleotide sequence of any one of SEQ ID No.: 1, SEQ ID No.: 2, SEQ ID No.: 15 or SEQ ID No.: 16, SEQ ID No.:19 or SEQ ID No.20;
c) identifying a XylT DNA fragment from said Nicotiana species or cultivar by performing a PCR using said genomic DNA or said cDNA and said primers, or by performing hybridization using said genomic DNA or said cDNA and said probes.

29. A method to isolate a Nicotiana XylT DNA fragment, comprising the steps of
a) identifying said Nicotiana XylT DNA fragment according to the method of claim 28; and
b) isolating said Nicotiana XylT DNA fragment.

30. A method to identify a Nicotiana XylT allele correlated with a low level of beta-1,2-xylose residues on protein-bound N-glycans comprising the steps of
(a) providing a population, optionally a mutagenized population, of different plant lines of a Nicotiana species or cultivar;
(b) identifying in each plant line of said population a Nicotiana XylT allele according to the method of claim 28;
(c) analyzing the level of beta-1,2-xylose residues on protein-hound N-glycans of each plant line of said population and identifying those plant lines having a lower level of beta-1,2-xylose residues on protein-bound N-glycans than other plant lines;
(d) correlating the low level of beta-1,2-xylose residues on protein-bound N-glycans in a plant line to the presence of a specific Nicotiana XylT allele.

31. A method to obtain a Nicotiana plant cell or plant with a low level of beta-1,2-xylose residues on protein-bound N-glycans, comprising the steps of
a) identifying a Nicotiana XylT allele correlated with a low level of beta-1,2-xylose residues on protein-bound N-glycans according to the method of claim 30;
b) introducing said Nicotiana XylT allele into a Nicotiana plant line of choice.

32. An isolated DNA fragment encoding a protein comprising the amino acid sequence of SEQ ID No.: 12, or SEQ ID No.:14, or any part thereof encoding at least one Nicotiana benthamiana-specific XylT amino acid.

33. The isolated DNA fragment of claim 32, comprising the nucleotide sequence of SEQ ID No.: 11, SEQ ID No.: 13, or SEQ ID No.: 21, or any part thereof comprising at least one Nicotiana benthamiana-specific XylT nucleotide.

34. An isolated DNA fragment encoding a protein comprising the amino acid sequence of SEQ ID No.: 4 or SEQ ID No.:6, SEQ ID No.: 8, SEQ ID No.:10, or any part thereof encoding at least one Nicotiana tabacum cv. Petite Havana SR1-specific XylT amino acid.

35. The isolated DNA fragment of claim 34, comprising the nucleotide sequence of SEQ ID No.: 3 or SEQ ID No.:5, SEQ ID No.: 7, SEQ ID No.:9, or SEQ ID No.: 17, or any part thereof comprising at least one Nicotiana tabacum cv. Petite Havana SR1-specific XylT nucleotide.

36. An isolated DNA fragment obtainable by the method of claim 28, encoding at least one Nicotiana species- or Nicotiana cultivar-specific XylT amino acid.

37. The isolated DNA fragment of claim 36, comprising at least one Nicotiana species- or Nicotiana cultivar-specific XylT nucleotide.

38. A chimeric gene comprising the following operably linked DNA fragments:
a) a plant expressible promoter;
b) a transcribable DNA region comprising
i) a first DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in antisense orientation;
ii) a second DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in sense orientation,
whereby an RNA molecule produced by transcription of said transcribable DNA region is capable of forming a double stranded RNA region by base-pairing at least between an RNA region corresponding to said first DNA region and an RNA region corresponding to said second RNA region; and
c) a DNA region comprising a transcription termination and polyadenylation signal functional in plants.

39. A chimeric gene comprising the following operably linked DNA fragments:
a) a plant expressible promoter;
b) a DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in sense orientation; and
c) a DNA region comprising a transcription termination and polyadenylation signal functional in plants.

40. A chimeric gene comprising the following operably linked DNA fragments
a) a plant expressible promoter;
b) a DNA region comprising at least 19 out of 20 consecutive nucleotides from a nucleotide sequence encoding a Nicotiana XylT protein, or the complement thereof, wherein said 19 out of 20 consecutive nucleotides encode at least one Nicotiana species- or cultivar-specific XylT amino acid, or from the nucleotide sequence of a Nicotiana XylT gene or a Nicotiana XylT cDNA, or the complement thereof, wherein said 19 out of 20 consecutive nucleotides comprise at least one Nicotiana species-specific XylT nucleotide, in antisense orientation; and
c) a DNA region comprising a transcription termination and polyadenylation signal functional in plants.

41. The chimeric gene of claim 38, wherein said Nicotiana species-specific XylT amino acid is a Nicotiana benthamiana-specific XylT amino acid.

42. The chimeric gene of claim 38, wherein said nucleotide sequence encoding a Nicotiana XylT protein comprises a nucleotide sequence encoding the amino acid sequence of SEQ ID No.: 12 or SEQ ID No.:14.

43. The chimeric gene of claim 38, wherein said Nicotiana species-specific XylT nucleotide is a Nicotiana benthamiana-specific XylT nucleotide.

44. The chimeric gene of claim 38, wherein said nucleotide sequence of said Nicotiana XylT gene comprises the nucleotide sequence of SEQ ID No.: 11, SEQ ID No.:13, or SEQ ID No. 21.

45. The chimeric gene of claim 38, wherein said Nicotiana cultivar-specific XylT amino acid is a Nicotiana tabacum cv. Petite Havana SR1-specific XylT amino acid.

46. The chimeric gene of claim 38, wherein said nucleotide sequence encoding said Nicotiana XylT protein comprises a nucleotide sequence encoding the amino acid sequence of SEQ ID No.: 4, SEQ ID No.:6, SEQ ID No.: 8 or SEQ ID No.:10.

47. The chimeric gene of claim 38, wherein said Nicotiana cultivar-specific XylT nucleotide is a Nicotiana tabacum cv. Petite Havana SR1-specific XylT nucleotide.

48. The chimeric gene of claim 38, wherein said nucleotide sequence of said Nicotiana XylT gene or said Nicotiana XylT cDNA comprises the nucleotide sequence of SEQ ID No.: 3, SEQ ID No.: 5, SEQ ID No.: 8, SEQ ID No.:10, or SEQ ID No.: 17.

49. A Nicotiana plant cell comprising the chimeric gene of claim 38.

50. A Nicotiana plant consisting essentially of the Nicotiana plant cells of claim 49.

51. A Nicotiana plant cell or plant obtained by the method of claim 31.

52. A seed of a Nicotiana plant of claim 50.

53. (canceled)

54. (canceled)

55. (canceled)

56. (canceled)

57. (canceled)

58. (canceled)

59. (canceled)

60. (canceled)

61. The method of claim 1, wherein said nucleotide sequences are obtainable from said Nicotiana species or cultivar.

62. The method of claim 2, wherein said Nicotiana species is Nicotiana benthamiana.

63. The method of claim 4, wherein said Nicotiana species is Nicotiana benthamiana.

64. The method of claim 6, wherein said Nicotiana species is Nicotiana tabacum cv. Petite Havana SR1.

65. The method of claim 8, wherein said Nicotiana species is Nicotiana tabacum cv. Petite Havana SR1.

66. The method of claim 12, wherein said nucleotide sequences are obtainable from said Nicotiana species or cultivar.

67. The method of claim 13, wherein said nucleotide sequences are obtainable from said Nicotiana species or cultivar.

68. The method of claim 14, wherein said nucleotide sequences are obtainable from said Nicotiana species or cultivar.

69. The method of claim 15, wherein said nucleotide sequences are obtainable from said Nicotiana species or cultivar.

70. The method of claim 16, wherein said Nicotiana species is Nicotiana benthamiana.

71. The method of claim 18, wherein said Nicotiana species is Nicotiana benthamiana.

72. The method of claim 20, wherein said Nicotiana species is Nicotiana tabacum cv. Petite Havana SR1.

73. The method of claim 22, wherein said Nicotiana species is Nicotiana tabacum cv. Petite Havana SR1.

74. A seed of a Nicotiana plant of claim 51.


SEQUENCE LISTING (26 sequences)

SEQ ID NO: 1 - DNA, 37 bp, Artificial Sequence
  oligonucleotide XylF4
caccttgtttctctcttcgctctcaactcaatcactc

SEQ ID NO: 2 - DNA, 27 bp, Artificial Sequence
  oligonucleotide XylR4
tcgatcacaactggaggatccgcataa

SEQ ID NO: 3 - DNA, 1513 bp, Artificial Sequence
  XylTc2Nt
caccttgtttctctcttcgctctcaactcaatcactctctatctctacttctcttcccactctgatcacttccgtcacaaatccccccaaaaccactttcctaatacccaaaaccactattccctgtcggaaaaccaccatgataatttccactcttctgtcacttcccaatataccaagccttggccaattttgccctcctacctcccctggtctcagaatcctaatgtttctttgagatcgtgcgagggttacttcggtaatgggtttactctcaaagttgatcttctcaaaacttcgccggagcttcaccagaaattcggcgaaaacaccgtatccggcgacggcggatggtttaggtgttttttcagtgagactttgcagagttcgatttgcgagggaggtgctatacgaatgaatccggacgagattttgatgtctcgtggaggcgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgagacacaattggaggaaacatggaaagcactcttttcaagcctcacttatgctaagaactttagtggcccagtttgtttccgtcacgccgttctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcattgttggtgctcatggagcaggtctaactcacatagtttctgcagcaccaaaagctgtaatactagaaattataagcagcgaatataggcacccccattttgctctgattgcacaatggaaaggattggagtaccatcccatatatttggaggggtcttatgcggatcctccagttgtgatga

SEQ ID NO: 4 - protein, 502 aa, Artificial Sequence
  Synthetic Construct
VSLFALNSITLYLYFSSHSDHFRHKSPQNHFPNTQNHYSLSENHHDNFHSSVTSQYTKPWPILPSYLPWSQNPNVSLRSCEGYFGNGFTLKVDLLKTSPELHQKFGENTVSGDGGWFRCFFSETLQSSICEGGAIRMNPDEILMSRGGEKLESVIGRSEDDELPVFKNGAFQIKVTDKLKIGKKLVDEKILNKYLPEGAISRHTMRELIDSIQLVGADEFHCSEWIEEPSLLITRFEYANLFHTVTDWYSAYVASRVTGLPSRPHLVFVDGHCETQLEETWKALFSSLTYAKNFSGPVCFRHAVLSPLGYETALFKGLTETIDCNGASAHDLWQNPDDKRTARLSEFGEMIRAAFGFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRHPHFALIAQWKGLEYHPIYLEGSYADPPVVM

SEQ ID NO: 5 - DNA, 1495 bp, Artificial Sequence
  XylTc7Nt
cacccttgtttctctcttcgctctcaactcaatcactctctatctctacttctcttcccaccctgatcacttccgccacaaatcccgccaaaaccacttttccttgtcggaaaaccgccatcataatttccactcttcaatcacttctcaatattccaagccttggcctattttgccctcctacctcccttggtctcaaaaccctaatgttgtttggagatcgtgcgagggttacttcggtaatgggtttactctcaaagttgaccttctcaaaacttcgccggagtttcaccggaaattcggcgaaaacaccgtctccggcgacggcggatggtttaggtgttttttcagtgagactttgcagagttcgatctgcgagggaggcgcaatacgaatgaatccggacgagattttgatgtctcgtggaggtgagaaattggagtcggttattggtaggagtgaagatgatgagctgcccgtgttcaaaaatggagcttttcagattaaagttactgataaactgaaaattgggaaaaaattagtggatgaaaaattcttgaataaatacttaccggaaggtgcaatttcaaggcacactatgcgtgagttaattgactctattcagttggttggcgccgatgattttcactgttctgagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgagacacaattggaggaaacatggaaagcacttttttcaagcctcacttatgctaagaactttagtggcccagtttgtttccgtcatgccgccctctcgcctttgggatatgaaactgccctgtttaagggactgtcagaaactatagattgtaatggagcttctgcccatgatttgtggcaaaatcctgatgataagaaaactgcacggttgtccgagtttggggagatgattagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcttctgtcatagttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgaa

SEQ ID NO: 6 - protein, 497 aa, Artificial Sequence
  Synthetic Construct
LVSLFALNSITLYLYFSSHPDHFRHKSRQNHFSLSENRHHNFHSSITSQYSKPWPILPSYLPWSQNPNVVWRSCEGYFGNGFTLKVDLLKTSPEFHRKFGENTVSGDGGWFRCFFSETLQSSICEGGAIRMNPDEILMSRGGEKLESVIGRSEDDELPVFKNGAFQIKVTDKLKIGKKLVDEKFLNKYLPEGAISRHTMRELIDSIQLVGADDFHCSEWIEEPSLLITRFEYANLFHTVTDWYSAYVASRVTGLPSRPHLVFVDGHCETQLEETWKALFSSLTYAKNFSGPVCFRHAALSPLGYETALFKGLSETIDCNGASAHDLWQNPDDKKTARLSEFGEMIRAAFGFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPVVIE

SEQ ID NO: 7 - DNA, 3408 bp, Artificial Sequence
  XylTg1Nt
caccttgtttctctcttcgctctcaactcaatcactctctatctctacttctcttcccactctgatcacttccgtcacaaatccccccaaaaccactttcctaatacccaaaaccactattccctgtcggaaaaccaccatgataatttccactcttctgtcacttcccaatataccaagccttggccaattttgccctcctacctcccctggtctcagaatcctaatgtttctttgagatcgtgcgagggttacttcggtaatgggtttactctcaaagttgatcttctcaaaacttcgccggagcttcaccagaaattcggcgaaaacaccgtatccggcgacggcggatggtttaggtgttttttcagtgagactttgcagagttcgatttgcgagggaggtgctatacgaatgaatccggacgagattttgatgtctcgtggaggcgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgaggttagattttgaaattttgcttgatctttaaattaaaggtttgaactttgtgaatgttggcagatatggaatacaataatggattttgtttgatctgtttaatgaagattgtctagaacctcattgttataaatatggtttgtttgcttcattaattaaagagcattccttaaaatctcgactagatgccagataacaccagttagttgacttttggattatggattttttttcatttaatcagataagatagtcattcttaaatgtttcactaaagaatttgtcatgatttcagtgtatatctttaagtgtatttggaattttggatttggatctagtactgaatgggtaactgcacttgtactccccagtcatctggggaggagcaacagattaaattcaagggttgaaaagtaatacagagtcagaaattaaccacaagttggaaaatggtaaatgtatgtggtctaagatgattactcctataacttttgatgtctaacatggagaaagttagttgatttatgctctttacttttccctttattgattttggttttcaaattctatcaattcctttgtttgattgctactcagattgaaccttagacggagtagcaatagaaaagtgaagaaaagccattttttctcctttcatctctttatttctgttttacacacagaatatggtagcatctgtctgaactagttaattttattccttaaaatttgcataactaattcgagtaaatgccttttgaagctttagttgtacaactggttgttgcattttgaggactatcgacttgatttgacagtgtctgatacatggcttgtaagttatgaaaacttatatctaggaagaaatcccaaccagagatagggagctgtcgcttggttatgagctactggctcaaagttcgagtttgaccagttaattttagatcttcggagtctaatcaaattctgaagcagtattgtgcactaataagaggaacacatcaaggatgtagcactgccaggttatgttaccttatttactaatgattgagaaccagcttaaatgatgacaaatggtcttagatttgttttttacattgctcatgactttgggatatttctggatcaacattttccagttctttatgtacttatcaaaaaattatccctgctagagcctagatgttagtgttcaagcaaccatgctagcttttaaggaagctccttctttgattcatgccatctttccgtaatcgatgccttacgttactgtcatttttctaattttcatttcagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgaggtatgtttgaaagtattgataacgatggcatgcattgtactgtgttatggatgaaagaaatgaaatcagcaattattttctagcaggcaatgctcttgagatgcttgtgtcaaattggtcagacttaatcctgagtttccatttgtttcagctttctgtttgactgactacaataattgtcccaatacctagttgttccggttggctcattcttacttctatttacgtgtcactgtttctctgaatggtccctttgtggtgaaaagtgcttttgctatgaagaaaaactagcaaagatttcatttctgggacaatttatttttaccttacatcacgtctcataaaattgcttcctattgcatactttaattcttggagagatgctttaatgtgaagaaagttctttcactccacttttaattcttggagagatgctttaatgtgaagaaagttctttcactccactactgtaagcttgctgcatgaattttacttggccatattgggggcgtgttttgatttatcttcaaattcattttcttcatgtaattcttttgagtaatttttttttctgttttctgtttgggaaaaaattcagacacaattggaggaaacatggaaagcactcttttcaagcctcacttatgctaagaactttagtggcccagtttgtttccgtcacgccgttctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcattgtcggcgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcga

SEQ ID NO: 8 - protein, 502 aa, Artificial Sequence
  Synthetic Construct
VSLFALNSITLYLYFSSHSDHFRHKSPQNHFPNTQNHYSLSENHHDNFHSSVTSQYTKPWPILPSYLPWSQNPNVSLRSCEGYFGNGFTLKVDLLKTSPELHQKFGENTVSGDGGWFRCFFSETLQSSICEGGAIRMNPDEILMSRGGEKLESVIGRSEDDELPVFKNGAFQIKVTDKLKIGKKLVDEKILNKYLPEGAISRHTMRELIDSIQLVGADEFHCSEWIEEPSLLITRFEYANLFHTVTDWYSAYVASRVTGLPSRPHLVFVDGHCETQLEETWKALFSSLTYAKNFSGPVCFRHAVLSPLGYETALFKGLTETIDCNGASAHDLWQNPDDKRTARLSEFGEMIRAAFGFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPVVI

SEQ ID NO: 9 - DNA, 3385 bp, Artificial Sequence
  XylTg3Nt
caccttgtttctctcttcgctctcaactcaatcactctctatctctacttctcttcccaccctgatcacttccgccacaaatcccgccaaaaccacttttccttgtcggaaaaccgccatcataatttccactcttcaatcacttctcaatattccaagccttggcctattttgccctcctacctcccttggtctcaaaaccctaatgttgtttggagatcgtgcgagggttacttcggtaatgggtttactctcaaagttgaccttctcaaaacttcgccggagtttcaccggaaattcggcgaaaacaccgtctccggcgacggcggatggtttaggtgttttttcagtgagactttgcagagttcgatctgcgagggaggcgcaatacgaatgaatccggacgagattttgatgtctcgtggaggtgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgaggttagattttgaaattttgcttgatctttaaattaaaggtttgaactttgtgaatgttggcagatatggaatacaataatggattttgtttgatctgtttaatgaagattgtctagaacctcattgttataaatatggtttgtttgcttcattaattaaagagcattccttaaaatctcgactagatgccagataacaccagttagttgacttttggattatggattttttttcatttaatcagataagatagtcattcttaaatgtttcactaaagaatttgtcatgatttcagtgtatatctttaagtgtatttggaattttggatttggatctagtactgaatgggtaactgcacttgtactccccagtcatctggggaggagcaacagattaaattcaagggttgaaaagtaatacagagtcagaaattaaccacaagttggaaaatggtaaatgtatgtggtctaagatgattactcctataacttttgatgtctaacatggagaaagttagttgatttatgctctttacttttccctttattgattttggttttcaaattctatcaattcctttgtttgattgctactcagattgaaccttagacggagtagcaatagaaaagtgaagaaaagccattttttctcctttcatctctttatttctgttttacacacagaatatggtagcatctgtctgaactagttaattttattccttaaaatttgcataactaattcgagtaaatgccttttgaagctttagttgtacaactggttgttgcattttgaggactatcgacttgatttgacagtgtctgatacatggcttgtaagttatgaaaacttatatctaggaagaaatcccaaccagagatagggagctgtcgcttggttatgagctactggctcaaagttcgagtttgaccagttaattttagatcttcggagtctaatcaaattctgaagcagtattgtgcactaataagaggaacacatcaaggatgtagcactgccaggttatgttaccttatttactaatgattgagaaccagcttaaatgatgacaaatggtcttagatttgttttttacattgctcatgactttgggatatttctggatcaacattttccagttctttatgtacttatcaaaaaattatccctgctagagcctagatgttagtgttcaagcaaccatgctagcttttaaggaagctccttctttgattcatgccatctttccgtaatcgatgccttacgttactgtcatttttctaattttcatttcagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgaggtatgtttgaaagtattgataacgatggcatgcattgtactgtgttatggatgaaagaaatgaaatcagcaattattttctagcaggcaatgctcttgagatgcttgtgtcaaattggtcagacttaatcctgagtttccatttgtttcagctttctgtttgactgactacaataattgtcccaatacctagttgttccggttggctcattcttacttctatttacgtgtcactgtttctctgaatggtccctttgtggtgaaaagtgcttttgctatgaagaaaaactagcaaagatttcatttctgggacaatttatttttaccttacatcacgtctcataaaattgcttcctattgcatactttaattcttggagagatgctttaatgtgaagaaagttctttcactccacttttaattcttggagagatgctttaatgtgaagaaagttctttcactccactactgtaagcttgctgcatgaattttacttggccatattgggggcgtgttttgatttatcttcaaattcattttcttcatgtaattcttttgagtaatttttttttctgttttctgtttgggaaaaaattcagacacaattggaggaaacatggaaagcactcttttcaagcctcacttatgctaagaactttagtggcccagtttgtttccgtcacgccgttctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcattgttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatc

SEQ ID NO: 10 - protein, 495 aa, Artificial Sequence
  Synthetic Construct
VSLFALNSITLYLYFSSHPDHFRHKSRQNHFSLSENRHHNFHSSITSQYSKPWPILPSYLPWSQNPNVVWRSCEGYFGNGFTLKVDLLKTSPEFHRKFGENTVSGDGGWFRCFFSETLQSSICEGGAIRMNPDEILMSRGGEKLESVIGRSEDDELPVFKNGAFQIKVTDKLKIGKKLVDEKILNKYLPEGAISRHTMRELIDSIQLVGADEFHCSEWIEEPSLLITRFEYANLFHTVTDWYSAYVASRVTGLPSRPHLVFVDGHCETQLEETWKALFSSLTYAKNFSGPVCFRHAVLSPLGYETALFKGLTETIDCNGASAHDLWQNPDDKRTARLSEFGEMIRAAFGFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPVVI

SEQ ID NO: 11 - DNA, 3298 bp, Artificial Sequence
  XylTg14Nb
caccttgtttctctcttcgctctcaactcaatcactctctatctctacttctcttcccaccctgatcactctcgtcgcaaatccccccagaaccacttttcctcgtcggaaaaccaccatcataatttccactcttcaatcacttcccaatattccaggccttggcctattttgccctcctacctcccttggtctcaaaaccctaatgttgcttggagatcatgcgagggttacttcggtaatggttttactctcaaagttgatcttctcaaaacttcgccggagcttcaccggaaattcggcgaaaacaccgtcttcggagacggcggatggtttaggtgtttcttcagtgagactttgcagagttcgatctgcgagggaggcgcaatacgaatgaatccagacgagattttgatgtctcgtggaggtgagaaattggagtcggttattggtaggagtgaagatgatgaggtgcccgcgttcaaaactggagcttttcagattaaagttactgataaactgaaatttgggaaaaaattagtggatgaaaacttcttgaataaatacttaccggaaggtgcaatttcaaggcacactatgcgtgagttaatcgactctattcagttggttggcgccaatgattttcactgttctgaggttagatttttgaaattttgtttgctctttaaattaaaggtttgaactttgtgaatgttggcagatatagaatacaataatggaatttgcttgatctgtttaatgaagattgtctggaacctcaatgctataaatatttgtttgtttgcttcattaattaaagagaatatcccaactagatgccagataacaccagttagttgacttttggatcggattgcatttcatttaatcagatatggtactcattcttaaatgtttcactaaagtatttgtcaagatttcagagtttatatgtaggtgtatttggaattctggatttggatctagtattgaatggattactgaacttgtactccccagtcatctggggaggagcaacagattaacttcaagggttgaaaagtaatactgagtcagaagttaaccacttcaacttggaaaattgtaaatgtgtgtggtctaagatgattactctaacttttgaggtctaacatggagaaagttagttgatttatgctctttacttttccctttattgattttggcttttaaattctatcaattccattgtttgattgctactcaaattgaaccttagacggagtagcaatagcaaaaagtgaagaaaggccattttttttctcctttcatctcttaatttccgttttacacacagaatatggtagaatctgtttgaagctttagttgaatagttatacaactggttattgcattttgaggactatcgacttgatttgacactggacagtgtctgatacatggcttgtaagttatgagaacttctatctaggaagaaatcccaaccagagatagggagctgtcacttggctatgagttactggctcaaagttcgagtttgaccagttaattttagatcctcaccaggataacatttagagtctaatcaaattctgaagcagtattgtgcactaataagaggaacacatgaaggatgtagcactactaggttatgttaccttatttactaataatgactgacaaccagcttaattgatgacaaatggtcttatatttgccttttacattgctcatgacttgggatatttctgaatcagcatttttcagttctttatgtacttatcaaaaaattatccctgctagatgttagtgttcaagcaaccatgctagcatttaacgaagctccttctttgattcatgcgatctttccgtaatctatgccttacgttactgtcatttttctaattttcatttcagtggattgaggagccgtcacttttgattacacgatttgagtatgcaaaccttttccacacaattaccgattggtatagtgcatacgtggcatcgagggttactggcttgcccagtcggccacatttggtttttgtagatggccattgtgaggtatgtctgaaagtattgataacgatggcatgcattgtactgtcttatggatgaaagaaatgaaaccagcaattattttctagcaggcaatgctcttgagatgcttgtgtcaaattggtcagacttaatcctgagtttccatttgtttcagctttctgtgtgactgactacaataattgtcccgatacctaattgttgcagttggctcattcttatttctatttacgtgtcactgtttctctgaatggccctttgtggtgaaaagagcttttgatatgtaaaaaaactagcaaagatttcatttctggaacaatttctttttaccttacatcacgtgtcataaaattgcttctaactgtatactttaattcttggagagatgctttcatgtgaagaaagttctttcactccactactggaagcttgctgcatgaattttacttggccatattggggccgtgttttgatttatcttcaaattcattttcttcatgtagttctttcgagtaattttttttcctcttttctgtttgaaaaaatttcagacacaattggaggaaacatggaaagcacttttttcaagcctcacttatgctaagaactttagtggcccagtttgtttccgtcatgccgtcctctcgcctttgggatatgaaactgccctgtttaagggactgtcagaaactatagattgtaatggagcttctgctcatgatttgtggcaaaatcctgatgataagaaaactgcacggttatccgagtttggggagatgatcagggcagcctttggatttcctgttgatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttaaaccactcggagtgcaaattaaatgtaattagtggattgtttgcccacatgtccatgaaagagcaagttcgagcaatccaagatgcttctgtcattgttggtgctcatggagcaggtctaacccacatagtttctgcagcaccaaaagctgtaatactagaaattataagcagcgaatataggcgcccccattttgctctgattgctcaatggaaaggattggagtaccatcccatatatttggaggggtcttatgcggatcctccagttgtgatcga

SEQ ID NO: 12 - protein, 493 aa, Artificial Sequence
  Synthetic Construct
VSLFALNSITLYLYFSSHPDHSRRKSPQNHFSSSENHHHNFHSSITSQYSRPWPILPSYLPWSQNPNVAWRSCEGYFGNGFTLKVDLLKTSPELHRKFGENTVFGDGGWFRCFFSETLQSSICEGGAIRMNPDEILMSRGGEKLESVIGRSEDDEVPAFKTGAFQIKVTDKLKFGKKLVDENFLNKYLPEGAISRHTMRELIDSIQLVGANDFHCSEWIEEPSLLITRFEYANLFHTITDWYSAYVASRVTGLPSRPHLVFVDGHCETQLEETWKALFSSLTYAKNFSGPVCFRHAVLSPLGYETALFKGLSETIDCNGASAHDLWQNPDDKKTARLSEFGEMIRAAFGFPVDRQNIPRTVTGPNVLFVRREDYLAHPRHGGKVQSRLSNEEQVFDSIKSWALNHSECKLNVISGLFAHMSMKEQVRAIQDASVIVGAHGAGLTHIVSAAPKAVILEIISSEYRRPHFALIAQWKGLEYHPIYLEGSYADPPV

SEQ ID NO: 13 - DNA, 3574 bp, Artificial Sequence
  XylTg19Nb
caccttgtttctctcttcgctctcaactcaatcactctctatctctacttctcttcccaccctgatcacaaatccccccaaaaccacttttccttgtcggaaaaccaccatcataatttccactcttcaatcacttctcaatattccaagccttggcctattttgccctcctacctcccttggtctcaaaaccctaatgttgcttggagatcgtgcgagggttacttcggtaatgggtttactctcaaagttgaccttctcaaaacttcgccggagtttcaccggaaattcggcgataacaccgtctccggtgacggcggatggtttaggtgttttttcagtgagactttgcagagttcgatctgcgagggaggcgcaatacgaatgaatccggacgatattttgatgtctcgtggaggtgagaaattggagtcggttattggtaggaatgaagatgatgagctgcccatgttcaaaaatggagctttccaaattgaagttactgataaactgaaaattgggaaaaaactagtggataaaaaattcttgaataaatacttaccgggaggtgcgatttcaaggcacactatgcgtgagttaattgactctattcagttggttggcgccgatgaatttcactgttctgaggttagattttgatatttatttgatctttaaattagaggtttgaactttgttaatgttggcagatatggaatacaataatggattttgtttgatctgtttaatgaagattgtctaaaacctcaatgctataaatatttgtttgtttgcttcattaattaaagagaatatcccgactagatgccagataacaccagttagttgacttttggattgggttgcatttcatttaatcagatatggtactcattcttaaatgtttcactaaagaatttgtcaagatttcagagtttatatataggtgtatttggaattctggatttggatctagtattgaatggattactgaatttgtactccccagtcatcaggggaggagcaatagatcgaattcaagggttgaaaagtaatactgagtcagaaattaaccactttaacttggaaacggtaaatgtatgtgttctaagatgattattcctataacttttgatgtctaatatggagaaagtgagttgatttatgctttttccttttccctttattgatgttggtttttaaattctatcaattcctttgtttggttgctactcaaattgaaccttagacggagtagcaatagcaaaaagtgaagaaaggacatttttttctcctttcatctctttatttccgtttgacatacagaatacggtagcatctgcctgaagtggttaatttcattccttaaaatttgcataactaatatttccgtttttgtttttgtttatcttttccattggcatgccatgttatttttggtttaggtttacataattatttatgtgatttctgatggagttactaatgattttttgtttttgtttttgtttttttcttttccttttcctgagtcgagggtcgattggaaatagcctctctgcccttttggataggggtaaggcctgggtacgtgtaccatccccagaccccactctgtgggactataccgggtagttgttgttgttgtaattcgagtaaatgccttttgaacctttagttgaatagttgtacaactggttgttgcattttgaggactatcgacttgatttgacactttacatgaaaacttttatctaggaagaaatccctaccagagatagggagctgtcgcttggttatgagctactggcttaaagtttgagtttgacctattaattttagatcttcaccaggataacatctagagtttaattaaattctcaagcagtattttgcactaataaggggaacacatgaaggatgtagcactactacgttatgttctttatttactattgattgacaaccagcttaaatgatgacaaatggtcttatatttgcttttttacattgctcatgacttgggatatttttgaatcaacatttttcggttctttatgtacttatcaaaaaattatccctgctagatgttagtgttcaagcaacatgctagcttttaaggaagctccttctttgattcatgccatctttccgaagccttacgtttctgtcatttttctaattttcatttcagtgggttgaggagccgtcacttttgattacacgatttgagtatgcaaaccttttccacacagttaccgattggtatagtgcatacgcggcatccagggttactggtttgcccagtcggccaaatttggtttttgtagatggccattgtgaggtatgtttgacagtattgataacgatggcatgcattgtactgtgttatggatgaaagaaatgaaaccatcaattattttctagtaggcaatgctcttaagatgcttgtgtcaaattggttagagttaatcctaagtttccatttgtttgagctttctgtttgactgactacaatacttgtcccaatacctagttgttgcggttggctcattcttacttctatttacgtgtcactgtttctctgaatggtccctttgtggtgaaaagagcttttgctatgtagaaaaactagcaaagatttcatttctggagcaacttatttttaccttacatcacgtctcataaaattgcttctaactgtatactttaattcttggagagatgctttcatgtgaataaagttctttcactccactactggaagcttgctgcatgaaatttacttggccatactggggccgtgttttgatttgtcttcaaattcattttcttcatgtagttctttcgagtaatattttttcctcttctgtttgaaaaaaattcagacacaattggaggaaacatggaaagcacttttttcaagcctcacttatgctaagaactttagtggcccagtttgtttccgtcatgctgtcctctcgcctttaggatatgaaactgccctgtttaagggactgtcagaaactatagattgtaatggagcttctgctcatgatttgtggcaaaagcctgatgataaaaaaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagctagtatttgattccataaagagctgggccttgaaccactcggagtgtaaattaaatgtaattaacggattgtttgcccacatgtccatgaaagagcaagttcgagcaatccaagatgcttctgtcattgttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcga

SEQ ID NO: 14 - protein, 490 aa, Artificial Sequence
  Synthetic Construct
VSLFALNSITLYLYFSSHPDHKSPQNHFSLSENHHHNFHSSITSQYSKPWPILPSYLPWSQNPNVAWRSCEGYFGNGFTLKVDLLKTSPEFHRKFGDNTVSGDGGWFRCFFSETLQSSICEGGAIRMNPDDILMSRGGEKLESVIGRNEDDELPMFKNGAFQIEVTDKLKIGKKLVDKKFLNKYLPGGAISRHTMRELIDSIQLVGADEFHCSEWVEEPSLLITRFEYANLFHTVTDWYSAYAASRVTGLPSRPNLVFVDGHCETQLEETWKALFSSLTYAKNFSGPVCFRHAVLSPLGYETALFKGLSETIDCNGASAHDLWQKPDDKKTARLSEFGEMIRAAFGFPVDRQNIPRTVTGPNVLFVRREDYLAHPRHGGKVQSRLSNEELVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPV

SEQ ID NO: 15 - DNA, 27 bp, Artificial Sequence
  oligonucleotide XylF8
cacctctcgcctttgggatatgaaact

SEQ ID NO: 16 - DNA, 24 bp, Artificial Sequence
  oligonucleotide XylR8
acagcttttggtgctgcagaaact

SEQ ID NO: 17 - DNA, 473 bp, Artificial Sequence
  XylTi4Nt
cacctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcattgttggtgctcatggagcaggtctaactcacatagtttctgcagcaccaaaagctgt

SEQ ID NO: 18 - DNA, 7954 bp, Artificial Sequence
  T-DNA of pTKW20
tcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatcgacctgcaggcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagacaagtttgtacaaaaaagcaggctccgcggccgcccccttcacctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcattgttggtgctcatggagcaggtctaactcacatagtttctgcagcaccaaaagctgtaagggtgggcgcgccgacccagctttcttgtacaaagtggtctagaggatccaagcttgtgcaggtaaatttctagtttttctccttcattttcttggttaggacccttttctctttttatttttttgagctttgatctttctttaaactgatctattttttaattgattggttatggcgcaaatattacatagctttaactgataatctgattactttatttcgtgtgtctatgatgatgatgataactgcagcgcaagcttatcgatttcgaacccagcttcccaactgtaatcaatccaaatgtaagatcaatgataacacaatgacatgatctatcatgttaccttgtttattcatgttcgactaattcatttaattaatagtcaatccatttagaagttaataaaactacaagtattatttagaaattaataagaatgttgattgaaaataatactatataaaatgatagatcttgcgctttgttatattagcattagattatgttttgttacattagattactgtttctattagtttgatattatttgttactttagcttgttatttaatattttgtttattgataaattacaagcagattggaatttctaacaaaatatttattaacttttaaactaaaatatttagtaatggtatagatatttaattatataataaactattaatcataaaaaaatattattttaatttatttattcttatttttactatagtattttatcattgatatttaattcatcaaaccagctagaattactattatgattaaaacaaatattaatgctagtatatcatcttacatgttcgatcaaattcattaaaaataatatacttactctcaacttttatcttcttcgtcttacacatcacttgtcatatttttttacattactatgttgtttatgtaaacaatatatttataaattattttttcacaattataacaactatattattataatcatactaattaacatcacttaactattttatactaaaaggaaaaaagaaaataattatttccttaccaagctggggtaccgaattcctcgagaccactttgtacaagaaagctgggtcggcgcgcccacccttacagcttttggtgctgcagaaactatgtgagttagacctgctccatgagcaccaacaatgacagatgcatcttggattgctcgaacttgctctttcatggacatgtgggcaaacaatccgttaattacatttaatttgcactccgagtggttcaaggcccagctctttatggaatcaaatacttgctcttcattgctaagcctagactgtacctttccaccatgacgtgggtgagctaaataatcctcacgtctaacaaagaggacattagggcctgtgactgtccttgggatgttctgtctatccacaggaaatccaaaggctgccctgatcatctccccaaactcggacaaccgtgcagttctcttatcatcaggattttgccacaaatcatgggcagaagctccattacaatctatagtttctgtcagtcccttaaacagggcagtttcatatcccaaaggcgagaggtgaagggggcggccgcggagcctgcttttttgtacaaacttgtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatctgcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgac

SEQ ID NO: 19 - DNA, 41 bp, Artificial Sequence
  oligonucleotide XylF9
ggccggatcctcgagacacaattggaggaaacatggaaagc

SEQ ID NO: 20 - DNA, 40 bp, Artificial Sequence
  oligonucleotide XylR9
ggccatcgatggtaccggcccagctctttatggaatcaaa

SEQ ID NO: 21 - DNA, 436 bp, Artificial Sequence
  XylTiNb
ggccggatcctcgagacacaattggaggaaacatggaaagcacttttttcaagcctcacttatgctaagaactttagtggcccagtttgtttccgtcatgccgtcctctcgcctttgggatatgaaactgccctgtttaagggactgtcagaaactatagattgtaatggagcttctgctcatgatttgtggcaaaatcctgatgataagaaaactgcacggttatccgagtttggggagatgatcagggcagcctttggatttcctgttgatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccggtaccatcgatggcc

SEQ ID NO: 22 - DNA, 5093 bp, Artificial Sequence
  T-DNA of pTKW29
aattacaacggtatatatcctgccagtactcggccgtcgaccgcggtaccccggaattaagcttgcatgcctgcaggtcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcaggactctagaggatcctcgagacacaattggaggaaacatggaaagcacttttttcaagcctcacttatgctaagaactttagtggcccagtttgtttccgtcatgccgtcctctcgcctttgggatatgaaactgccctgtttaagggactgtcagaaactatagattgtaatggagcttctgctcatgatttgtggcaaaatcctgatgataagaaaactgcacggttatccgagtttggggagatgatcagggcagcctttggatttcctgttgatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccggtaccatcgatttcgaacccaatttcccaactgtaatcaatccaaatgtaagatcaatgataacacaatgacatgatctatcatgttaccttgtttattcatgttcgactaattcatttaattaatagtcaatccatttagaagttaataaaactacaagtattatttagaaattaataagaatgttgattgaaaataatactatataaaatgatagatcttgcgctttgttatattagcattagattatgttttgttacattagattactgtttctattagtttgatattatttgttactttagcttgttatttaatattttgtttattgataaattacaagcagattggaatttctaacaaaatatttattaacttttaaactaaaatatttagtaatggtatagatatttaattatataataaactattaatcataaaaaaatattattttaatttatttattcttatttttactatagtattttatcattgatatttaattcatcaaaccagctagaattactattatgattaaaacaaatattaatgctagtatatcatcttacatgttcgatcaaattcattaaaaataatatacttactctcaacttttatcttcttcgtcttacacatcacttgtcatatttttttacattactatgttgtttatgtaaacaatatatttataaattattttttcacaattataacaactatattattataatcatactaattaacatcacttaactattttatactaaaaggaaaaaagaaaataattatttccttaccaattggggtaccggcccagctctttatggaatcaaatacttgctcttcattgctaagcctagactgtacctttccaccatgacgtgggtgagctaaataatcctcacgtctaacaaagaggacattagggcctgtgactgtccttgggatgttctgtctatcaacaggaaatccaaaggctgccctgatcatctccccaaactcggataaccgtgcagttttcttatcatcaggattttgccacaaatcatgagcagaagctccattacaatctatagtttctgacagtcccttaaacagggcagtttcatatcccaaaggcgagaggacggcatgacggaaacaaactgggccactaaagttcttagcataagtgaggcttgaaaaaagtgctttccatgtttcctccaattgtgtctcgagcgtgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttggtcttctgagactgtatctttgacatttttggagtagaccagagtgtcgtgctccaccatgttgacgaagattttcttcttgtcattgagtcgtaaaagactctgtatgaactgttcgccagtcttcacggcgagttctgttagatcctcgatttgaatcttagactccatgcatggccttagattcagtaggaactacctttttagagactccaatctctattacttgccttggtttatgaagcaagccttgaatcgtccatactggaatagtacttctgatcttgagaaatatgtctttctctgtgttcttgatgcaattagtcctgaatcttttgactgcatctttaaccttcttgggaaggtatttgatctcctggagattgttactcgggtagatcgtcttgatgagacctgctgcgtaggcctctctaaccatctgtgggtcagcattctttctgaaattgaagaggctaaccttctcattatcagtggtgaacatagtgtcgtcaccttcaccttcgaacttccttcctagatcgtaaagatagaggaaatcgtccattgtaatctccggggcaaaggagatctcttttggggctggatcactgctgggccttttggttcctagcgtgagccagtgggctttttgctttggtgggcttgttagggccttagcaaagctcttgggcttgagttgagcttctcctttggggatgaagttcaacctgtctgtttgctgacttgttgtgtacgcgtcagctgctgctcttgcctctgtaatagtggcaaatttcttgtgtgcaactccgggaacgccgtttgttgccgcctttgtacaaccccagtcatcgtatataccggcatgtggaccgttatacacaacgtagtagttgatatgagggtgttgaatacccgattctgctctgagaggagcaactgtgctgttaagctcagatttttgtgggattggaattaattcgtcgagcggccgctcgacgagctccctatagtgagtcgtattagaggccgacttgggggccggccatttaaatgaattcgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatcaaagatccggcccatgatccatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatctcacgcgtctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaagatccattaattcggtactcgaggcattacggcattacggcactcgcgagggtcccaattcgagcatggagccatttacaattgaatatatcctgccg

SEQ ID NO: 23 - DNA, 2318 bp, Nicotiana tabacum cv. Xanthi
  5 UTR (1)..(18)
  CDS (19)..(1590)
  3 UTR (1592)..(2291)
  polyA_signal (2292)..(2318)
agtcagagagagaagaagatgaacaagaaaaagctgaaatttcttgtttctctcttcgctctcaactcaatcactctctatctctacttctcttcccactctgatcacttccgtcacaaatccccccaaaaccactttcctaatacccaaaaccactattccctgtcggaaaaccaccatgataatttccactcttctgtcacttcccaatataccaagccttggccaattttgccctcctacctcccctggtctcagaatcctaatgtttctttgagatcgtgcgagggttacttcggtaatgggtttactctcaaagttgatcttctcaaaacttcgccggagcttcaccagaaattcggcgaaaacaccgtatccggcgacggcggatggtttaggtgttttttcagtgagactttgcagagttcgatttgcgagggaggtgctatacgaatgaatccggacgagattttgatgtctcgtggaggcgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgagacacaattggaggaaacatggaaagcactcttttcaagcctcacttatgctaagaactttagtggcccagtttgtttccgtcacgccgttctctcgcctttgggatatgaaactgccctgtttaagggactgacagaaactatagattgtaatggagcttctgcccatgatttgtggcaaaatcctgatgataagagaactgcacggttgtctgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcttctgtcatagttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgacaggctcagcagcattttgaggagtcttgggtgctaagtccgctcgacagtttgaatagttcggcttttctctaaaagacggggaaggatagaggaattcggggttctggaacttggagcctgggaattttgataaatatgtttcacacgcagttctgtagtcaatggttgcaatctaggtcctcaatctggtgttgataagcttggcaatttccagcagctactaatttattagcccgctctgactcggttatggactaccagagcaatcatatcaaatggaagcatggaatcctgattgtggaatggtgagctcattgaagagcatattctttatggtgttgaagattacaattcacaattaacacgtgtatgtgaaagattaggttggtacacttacttacaattcattgtcaattgtttttcattattctcattaatgatcataggataagaacatgagaaaaccatccatgttctgtgttgttttcccatcaatccggccaccctcttccctccttatgtagagatgatttcaacagagtttgttttgtagttgtaacacttgcactcccagttacagttttgcattcgacacattcatcccatcagatgtcaagtttaaaggcataagacatttgacatattgaagaagcagattaacacgaacgtcagtatgatgcttcagtgaagatatggttgtaacttgtaaccaaacaaaagaaatgagactttgacaaaaaaaaaaaaaaaaaaaaaaaaaaa

SEQ ID NO: 24 - protein, 523 aa, Nicotiana tabacum cv. Xanthi
MNKKKLKFLVSLFALNSITLYLYFSSHSDHFRHKSPQNHFPNTQNHYSLSENHHDNFHSSVTSQYTKPWPILPSYLPWSQNPNVSLRSCEGYFGNGFTLKVDLLKTSPELHQKFGENTVSGDGGWFRCFFSETLQSSICEGGAIRMNPDEILMSRGGEKLESVIGRSEDDELPVFKNGAFQIKVTDKLKIGKKLVDEKILNKYLPEGAISRHTMRELIDSIQLVGADEFHCSEWIEEPSLLITRFEYANLFHTVTDWYSAYVASRVTGLPSRPHLVFVDGHCETQLEETWKALFSSLTYAKNFSGPVCFRHAVLSPLGYETALFKGLTETIDCNGASAHDLWQNPDDKRTARLSEFGEMIRAAFGFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPVVIDRLSSILRSLGC

SEQ ID NO: 25 - DNA, 4973 bp, Nicotiana tabacum cv. Xanthi
  5 UTR (1)..(18)
  CDS (19)..(696)
  Intron (697)..(2028)
  CDS (2029)..(2178)
  Intron (2179)..(3606)
  CDS (3607)..(4326)
  3 UTR (4330)..(4973)
agtcagagagagaagaagatgaacaagaaaaagctgaaaattcttgtttctctcttcgctctcaactcaatcactctctatctctacttctcttcccaccctgatcacttccgccacaaatcccgccaaaaccacttttccttgtcggaaaaccgccatcataatttccactcttcaatcacttctcaatattccaagccttggcctattttgccctcctacctcccttggtctcaaaaccctaatgttgtttggagatcgtgcgagggttacttcggtaatgggtttactctcaaagttgaccttctcaaaacttcgccggagtttcaccggaaattcggcgaaaacaccgtctccggcgacggcggatggtttaggtgttttttcagtgagactttgcagagttcgatctgcgagggaggcgcaatacgaatgaatccggacgagattttgatgtctcgtggaggtgagaaattggagtcggttattggtaggagtgaagatgatgagctgcccgtgttcaaaaatggagcttttcagattaaagttactgataaactgaaaattgggaaaaaattagtggatgaaaaattcttgaataaatacttaccggaaggtgcaatttcaaggcacactatgcgtgagttaattgactctattcagttggttggcgccgatgattttcactgttctgaggttagattttgaattttgtttgctctttaaattaaaggtttaaactttgtgaatgttggcagatatggaatacactaatggattttgtttgatctgtttaatgaagattgtctagaacctcaatgttataaatatggtttggttgcttcattaattaaagagaattccttaatatcccgactagatgccagataacaccagttagttgacttttggattattggttgcatttcatttgatcagataaattgttcattcttaaatgtttcactaaagaattactcaagatttcagagtttatatgtaggtgtatgtatttggaattctggatttggatctagtattgaatggattactgaacttgtactccccagtcatctggggaggagcaatagatcaaattcaagggttgaaaagtaatactgagtcagaaattaaccactttaacttggaaaacggtaaatgtatgtgttctaagatggttattcctataacttttgatgtctaatatggagaaagtgagttgatttatgctttttccttttccctttattggtgttggtttttaaattctatcaattcctttgtttgattgctactcaaattgaaccttagacggagtagcaatagcaaaaagtgaaggccattcttttctcctttcatctctttatttccgtttgacatacagaatatggtagcatctgtctgaagtggttaattttattccttaaaatttgcataactaattcgagtaaatgccttttgaagctttagttgaatagttctacaactggttgttgcattttgaggactatcgacttgatttgacacttgacattgtctgatacatggcttgtaagttatgaaaacttttatctaggaagaaatcccaaccagagatagggagctgtcacttggttatgagctactggctcaaagttcaagtttgaccagttaattttagatcttcaccaggataacatttagagtctaatcaaattctgaagcagtattgtgcactaataagaggaacacatgaaggatgtagcactactaggttatgttaccttatttactaatgattgacaaccagcttaaatgatgacaaatagtcttatatttgctttttcacattgctcatgacttgggatatttccgaatcaacatattttagttctttatgtacttaactacttatcaaaaaattatccctgctagatgttagtgttcaagcaaccatgctagcttttaaggaagctccttctttgattcatgccatctttccgaaatcgatgccttacgttactgtcatttttctaattttcatttcagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgaggtatgtttgaaagtattgataacgatggcatgcattgtactgtgttacggatgaaagaaatgaaaccagcaattattttctagcaggcaatgctcttgagatgcttgtgtcaaattggtcagacttaatcctgagtttccatttgtttcagctttctgtttgactgactacaataattgtcccaattaggggtgtcaatggatattcgaaagccgactaaaccgaccgaaccgtaccgtaccgatttttaggtttctttttaagaaaccgtaggtttttatataaatctataaccgcaccgataattagggtaggttttttattttataaaaataagccgaaaaaataccgaaccgtaccgaataaattttacatgtggaaaatatatttatttagtaagtttaaaaataataatgcattaaattttctttgggccatggaattatgaaaactattacaagccaacaagtaattagactcaaaatactaattcctaaaacctattatgttacttctacttaaactaagttatttcaagtatctttattagcaagacacaaagtattctagcgattatgagcaaactacaatgtattgaatatgtttcctttcatatattttagatttatctttttgaatatttaatcttctatagactctattcttgagtcccagcttggtatatctttcaactcgtgtgatttatattttctttgcctttgtttgatttcttttacgttgttgtagaatagtcgatggatctatactctagccatctttctttttctttttttaattcatcaccttttaaacagtaaaaatgtctaaagaatttttctaagtcctataaaagaacgtatgttattgcattctacttctactggtgaattttacatgatattaaaaaattaaccgaaccttaccgctaccgaagagaaaccgacatgattgggacggtttcgaaaagtctaattttggttatacataatagaataaccgaaaaattggtatggtacaaattttataaaataaccggccgaaccgaaccattgacacccctagtcccaatacctagttgttgcagtttgctcattcttacttctatttacgtcactgtttctctgaatggtccctttgtggtgaaaagagcttttgctatgtagaaaaactagcaatgatttcatagctgaaacaatttatttttaccttacatcatgtcttataaaattgcttctaactgtatactttaattcttggagagatgctttcatgtgaagaaagttctttcactccactactggaagcttgccgtatgaattttacttggccatattgtggccgtgctttgatttatcttcaaattcattttcttcatatagttctttcgagtaattcttttttcctcttttctgtttgaaaaaaattcagacacaattggaggaaacatggaaagcacttttttcaagcctcacttatgctaagaactttagtggcccagtttgtttccgtcatgccgccctctcgcctttgggatatgaaactgccctgtttaagggactgtcagaaactatagattgtaatggagcttctgcccatgatttgtggcaaaatcctgatgataagaaaactgcacggttgtccgagtttggggagatgattagggcagcctttagatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcttctgtcatagttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgacaagctcagcagcattttgaggagtcttgggtgctaaatctgctcgacagtttagttcgtcttttctctaaaagactgggaaggatagaggaattcggggttctggaacctggagcctgggaattgtgtaaaatatgtttcacacgcagttctatagtcaattgctgcaatctggtgttcataagcttggaaatttccagcagctactaacttattagcccactctgactcagttatggactaccagagcaatcatatcaaatgggagcatggaatcctgattgtggaatggtgagctcattgaagagcatattctttatggtgttgaagattacagttgacgagtaacacgtgtatgtgaaagattaggttgttacactttcttgcaattcattgtcaattgtttttcgtcattcttattaatgatcataggataagaacatgagaaaaccatccatgttctctgttgttttcccatcaatctggccaccctctttcctctttatgtagagatgatttcaacagagtttgttttgtagttgtaatacttgtactcacagttactgttttgcattcatcccatcagatgtcgaagaagcagattaacaagaacgtcagtatgatgtttcagtgaatatatggttgtaacttgtaaccaaacaaaagaaatgagactttgac

SEQ ID NO: 26 - protein, 516 aa, Nicotiana tabacum cv. Xanthi
MNKKKLKILVSLFALNSITLYLYFSSHPDHFRHKSRQNHFSLSENRHHNFHSSITSQYSKPWPILPSYLPWSQNPNVVWRSCEGYFGNGFTLKVDLLKTSPEFHRKFGENTVSGDGGWFRCFFSETLQSSICEGGAIRMNPDEILMSRGGEKLESVIGRSEDDELPVFKNGAFQIKVTDKLKIGKKLVDEKFLNKYLPEGAISRHTMRELIDSIQLVGADDFHCSEWIEEPSLLITRFEYANLFHTVTDWYSAYVASRVTGLPSRPHLVFVDGHCETQLEETWKALFSSLTYAKNFSGPVCFRHAALSPLGYETALFKGLSETIDCNGASAHDLWQNPDDKKTARLSEFGEMIRAAFRFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPVVIDKLSSILRSLGC